(19) United States
(12) Patent Application Publication
(10) Pub. No.: US 2020/0398068 A1
(43) Pub. Date: Dec. 24, 2020

(54) SENSORY STIMULATION APPARATUS

(71) Applicant: Augmented Bionics Pty Ltd, Milton, Queensland (AU)

(72) Inventors: Viraj AGNIHOTRI, Upper Mount Gravatt (AU); Mahantheshaiah Gangenapura CHANDRASHEKHARAIAH, St Lucia (AU); Ahmed Toaha TOAHA MOBASHSHER, St Lucia (AU); Amin ABBOSH, Mansfield (AU); Nicholas JABBOUR, Arana Hills (AU)

(21) Appl. No.: 16/979,130

(22) PCT Filed: Mar. 12, 2019
(86) PCT No.: PCT/AU2019/050219

§ 371 Date: Sep. 8, 2020

(30) Foreign Application Priority Data
Mar. 18, 2018 (AU) 2018900824

(51) Int. Cl. A61N 2/00; A61N 2/02; G16H 40/67; G06N 20/00

(52) U.S. Cl. CPC A61N 2/006; A61N 2/002; G06N 20/00; G16H 40/67; A61N 2/02

(57) ABSTRACT

Apparatus for providing sensory stimulation to a subject, the apparatus including an input that acquires input signals indicative of a stimulatory input, a signal generator, a coil system including at least one coil and an electronic controller operating in accordance with software instructions. In use, the controller receives the input signals from the input, performs analysis of the input signals and, uses results of the analysis to cause the signal generator to generate stimulation signals, the stimulation signals being applied to the coil system to thereby generate a stimulatory electromagnetic field in a target region of the subject, the stimulatory electromagnetic field being configured to selectively activate sensory neurons to thereby stimulate the subject in accordance with the stimulatory input.

### BACKGROUND OF THE INVENTION

The present invention relates to a method and apparatus for providing sensory stimulation, such as audible or visual stimulation, to a subject, and in one particular example, providing sensory stimulation by generating a stimulatory electromagnetic field to selectively activate sensory neurons.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Neuromodulation has multiple applications in modern medicine. Some widely known applications include prosthetics i.e. devices that improve impaired sensory, motor or cognitive neural functions, devices to regulate the body's organs in disease states through neural mechanisms, and investigation of neural functions in the peripheral and central nervous systems. These neuromodulation techniques influence the flow of ions through the neurons to either stimulate or block the firing of Action Potentials in the nerve. Traditionally, the main technique for neuromodulation has been the use of direct electric current which induces a potential or voltage gradient across the neuron. Once sufficient, this potential gradient allows the neuron to initiate or suppress an Action Potential depending on the intended effect. This technique was successfully demonstrated by the first cochlear implants. Another neuromodulation methodology has been the use of magnetic stimulation. Devices such as Transcranial Magnetic Stimulators use magnetic pulses to induce an electric field across the neuron, which leads to the potential gradient required for modulation. Other neuromodulation techniques include optogenetics, thermal, acoustic/mechanical, and chemical neuromodulation.

The World Health Organisation estimates that there are around 466 million people in the world that suffer from a disabling hearing loss. Moreover, the current production of hearing devices meets only 10% of this global need. Depending on the type and degree of hearing loss, different devices are prescribed to patients with hearing loss. There are four types of devices currently on the market that are used to enable hearing in patients with hearing loss. These include hearing aids, bone conduction hearing aids, middle ear implants, and cochlear implants, which are prescribed for patients suffering from severe to profound sensorineural hearing loss.

In this regard, the hearing system is made up of three parts where the outer ear or the pinna funnels the sound to the ear canal. The sound vibrations then fall upon the ear drum of the middle ear and are amplified through three tiny bones called the ossicles. The ossicles then transfer the vibrations into the cochlea placed in the inner ear. The cochlea is a spiral organ that resembles a snail's shell. Inside the cochlea is the organ of corti, which contains hair cells. These hair cells sense the vibrations of the sound and convert them into action potentials that get transferred to spiral ganglion neurons attached to them. The spiral ganglion cells then bundle up to form the auditory nerve.

If there is a problem in the middle ear, the patient suffers from a conductive hearing loss. If the problem is in the inner ear or subsequent auditory processing pathways in the brain, the patient suffers from sensorineural hearing loss.

Cochlear implants are prescribed when the hair cells of the patient are not working but the spiral ganglion neurons are still functional. In this case the implant is placed inside the cochlea and injects currents which depolarise the spiral ganglion neurons and leads to a series of action potentials or spikes. These spikes are then carried to the brain to be processed as auditory information.

As described in “Cochlear Implants: System Design, Integration, and Evaluation” by Fan-Gang Zeng, Senior Member, IEEE, Stephen Rebscher, William Harrison, Xiaoan Sun, and Haihong Feng in IEEE Reviews In Biomedical Engineering, VOL. 1, 2008, the cochlear implant system can be broken down into a number of parts. First, the external processor placed behind the ear with a hook and a battery case uses a microphone to pick up the sounds from the environment. These sound waves are converted from analog to digital signals after which they are processed and encoded into a radiofrequency (RF) signal. This RF signal is sent to an antenna in the headpiece. The headpiece is held in place by a magnet attracted to an internal receiver, which is placed under the skin behind the ear. A sealed stimulator contains active electronic circuits that derive power from the RF signal, decode it, convert it into electrical currents, and send them along wires leading to the cochlea. The electrodes at the end of the wires and inside the cochlea then stimulate the auditory nerve according to the sent electric signals.

Cochlear implants require a highly invasive surgery where the doctor makes an incision behind the ear, drills insides the temporal bone of the skull, pushes the internal receiver under the skull and the electrode inside the cochlea. This surgery subjects the patient to multiple risks like infection, facial paralysis, vertigo, loss of taste, tinnitus, insertion trauma, and other risks associated with anesthesia. Additionally, the procedure destroys any residual hearing, meaning there is a risk that the patients hearing will be worse, or at least lose some resolution, after the procedure. A further consequence of this is that the procedure is not reversible, meaning this cannot be used temporarily, such as an in cases of temporary hearing loss, or to allow direct interface with devices, for example for use in virtual reality applications or similar.

Transcranial Magnetic Stimulation (TMS) has been used for anti-depression therapy, as well as to map the functionality of different areas of the brain, to treat tinnitus, therapy for Parkinson's disease, Alzheimer's disease, and most recently to stimulate retinal neurons.

J. Y. Shin and J.-H. A, “Electrodeless, Non-Invasive Stimulation of Retinal Neurons Using Time-Varying Magnetic Fields,” IEEE Sensors Journal, vol. 16, no. 24, pp. 88832-8840, 2016 describes a surgically non-invasive retinal stimulation method by using time-varying magnetic fields. Retinal stimulations are achieved by inducing eddy currents on retinal ganglion cells with time-varying magnetic fields. The stimulator is developed using a voltage source, a voltage booster, a trigger circuit, a driver circuit, a storage capacitor bank, and a stimulating coil.

US20070260107 describes a system for Stereotactic Transcranial Magnetic Stimulation (sTMS) at predetermined locations with the brain or spinal cord and incorporates an array of electromagnets arranged in a specified configuration where selected coils in the array are pulsed simultaneously. Activation of foci demonstrated by functional MRI or other imaging techniques can be used to locate the neural region affected. Imaging techniques can also be utilized to determine the location of the designated targets.

US20080046053 describes an apparatus for generating focused currents in biological tissue. The apparatus comprises an electric source capable of generating an electric field across a region of tissue and means for altering the permittivity of the tissue relative to the electric field, whereby a displacement current is generated. The means for altering the permittivity may be a chemical source, optical source, mechanical source, thermal source, or electromagnetic source.

US2009/0156884 describes the treatment of specific neurological and psychiatric illnesses using Transcranial Magnetic Stimulation (TMS) requires that specific neuroanatomical structures are targeted using specific pulse parameters. Described herein are methods of positioning and powering TMS electromagnets to selectively stimulate a deep brain target region while minimizing the impact on non-target regions between the TMS electromagnet and the target. Use of these configurations may involve a combination of physical, spatial and/or temporal summation. Specific approaches to achieving temporal summation are detailed.

U.S. Pat. No. 8,972,004 describes devices and systems for the non-invasive treatment of medical conditions through delivery of energy to target tissue, comprising a source of electrical power, a magnetically permeable toroidal core, and a coil that is wound around the core. The coil and core are embedded in a continuous electrically conducting medium, which is adapted to have a shape that conforms to the contour of an arbitrarily oriented target body surface of a patient. The conducting medium is applied to that surface by any of several disclosed methods, and the source of power supplies a pulse of electric charge to the coil, such that the coil induces an electric current and/or an electric field within the patient, thereby stimulating tissue and/or one or more nerve fibers within the patient. The invention shapes an elongated electric field of effect that can be oriented parallel to a long nerve. In one embodiment, the device comprises two toroidal cores that lie adjacent to one another.

US2011/0029044 describes a system including a sensor device configured to sense a property of a mammal without physically contacting the mammal. The system also includes a signal generator configured to generate a signal indicative of the sensed property of the mammal. The system further describes a neuromodulation device configured to output a stimulus operable to modulate a nervous system component of the mammal in response to the signal indicative of the sensed property of the mammal.

US2013/0245486 describes devices and methods that treat a medical condition, such as migraine headache, by electrically stimulating a nerve noninvasively, which may be a vagus nerve situated within a patient's neck. Preferred embodiments allow a patient to self-treat his or her condition. Disclosed methods assure that the device is being positioned correctly on the neck and that the amplitude and other parameters of the stimulation actually stimulate the vagus nerve with a therapeutic waveform. Those methods comprise measuring properties of the patient's larynx, pupil diameters, blood flow within an eye, electrodermal activity and/or heart rate variability.

A major challenge in TMS is coil design, specifically creating coils that can appropriately stimulate deeper neuronal tissues whilst minimising stimulation in other areas, such as in the brain adjacent the scalp. This challenge is brought upon by the very physical laws that govern the electromagnetic fields as the magnetic field is inversely proportional to the square of the distance away from the coil. Some common designs of TMS coils include the circular coil, figure-of-8 coil, C-core coils, crown coil, and the H-coil. The suitability of the coil design depends on the application. For a surface level cortical stimulation, the circular and figure-of-8 coil are used, where the latter provides better focality. For deep brain stimulation (DBS) protocols, much bigger coil designs like the C-core, crown, and H-coils are used. The C-core coils also contain in them a high permeability iron core, which in turn strengthens the field, reduces heating, and minimises scalp stimulation.

### SUMMARY OF THE PRESENT INVENTION

In one broad form an aspect of the present invention seeks to provide an apparatus for providing sensory stimulation to a subject, the apparatus including: an input that acquires input signals indicative of a stimulatory input; a signal generator; a coil system including at least one coil; and, an electronic controller operating in accordance with software instructions that: receives the input signals from the input; performs analysis of the input signals; and, uses results of the analysis to cause the signal generator to generate stimulation signals, the stimulation signals being applied to the coil system to thereby generate a stimulatory electromagnetic field in a target region of the subject, the stimulatory electromagnetic field being configured to selectively activate sensory neurons to thereby stimulate the subject in accordance with the stimulatory input.

In one embodiment the input includes: an input sensor that senses the stimulatory input; and, a wireless transceiver that receives the input signal from a remote device.

In one embodiment the input sensor includes at least one of a microphone and an imaging device.

In one embodiment the stimulatory input is audible, and the sensory neurons are spiral ganglion neurons.

In one embodiment the stimulatory input is visual, and the sensory neurons are at least one of: retinal ganglion neurons; an optic nerve; a lateral geniculate nucleus; and a visual cortex.

In one embodiment the stimulatory electromagnetic field is generated to minimise a magnitude of the stimulatory electromagnetic field outside the target region.

In one embodiment the stimulatory electromagnetic field includes at least one of: a superposition of a plurality of electromagnetic fields; at least one inhomogeneous electromagnetic field; and, a sequence of electromagnetic fields.

In one embodiment the coil system includes at least one of: at least two coils; at least three coils; at least four coils; less than ten coils; less than eight coils; and, at least one primary coil and at least one secondary coil.

In one embodiment the coil system has a coil geometry arranged to focus electromagnetic fields from each of a plurality of coils on the target region.

In one embodiment different ones of the plurality of coils are focused on different parts of the target region.

In one embodiment the coil system includes a number of coils circumferentially spaced around an axis, the axis being coincident with the target region and the coils being arranged at an angle relative to the axis, so that ends of the coils face the target region.

In one embodiment the coil system includes at least one coil coincident with the axis.

In one embodiment the coil system includes at least one coil that is at least one of: a conical tapered coil; a dual lobe coil; a butterfly coil; a flat coil; a spiral coil; a helical coil; a multi layered helical coil; and, wound on a core.

In one embodiment at least one winding of at least one coil has at least one of: an inner radius of at least one of: at least 0.2 mm; at least 0.5 mm; at least 1 mm; at least 5 mm; at least 10 mm; less than 1.5 mm; less then 10 mm; less than 15 mm; and, less than 20 mm; and, an outer radius of at least one of: at least 5 mm; at least 8 mm; at least 10 mm; at least 20 mm; at least 30 mm; and, less than 50 mm; less than 60 mm.

In one embodiment the coil system includes at least one axial coil configured to generate an electric field in the target region.

In one embodiment the axial coil includes a plurality of conductors extending along an axis of a coil geometry and wherein the coil geometry has a shape that is at least one of: a cone; a hemisphere; a concave hemisphere; a convex hemisphere; and, a cylinder.

In one embodiment at least one coil is wound from a conductor at least one of: having a cross sectional area of at least one of: at least 0.001 mm²; at least 0.01 mm²; at least 0.1 mm²; at least 1 mm²; at least 5 mm²; at least 10 mm²; less than 20 mm²; and, less than 15 mm²; having a cross sectional shape of at least one of: round; and, rectangular; and, made from: a wire; a copper wire; and, a braided wire.

In one embodiment the coil is wound about a core that is at least one of: an air core; a soft magnetic composite core; an insulated magnetic core; a laminated core; a high permeability magnetic core; and, a metal core.

In one embodiment the core has at least one of: a radius of at least one of: at least 0.2 mm; at least 0.5 mm; at least 1 mm; at least 5 mm; at least 10 mm; less than 1.5 mm; less then 10 mm; less than 15 mm; and, less than 20 mm; and, a length of at least one of: at least 0.5 mm; at least 5 mm; at least 10 mm; at least 15 mm; about 20 mm-30 mm; and, less than 40 mm.

In one embodiment the core tapers inwardly proximate an end of the core closest to the subject.

In one embodiment apparatus includes at least one shield positioned adjacent the coil system to reduce stray fields.

In one embodiment the at least one shield includes: a diamagnetic shield; a conductive shield; a shield positioned adjacent each coil; and, a shield positioned adjacent each coil, each shield including an opening having a radius of at least one of: at least 0.2 mm; at least 0.5 mm; about 1 mm; and, less than 1.5 mm.

In one embodiment the apparatus includes a housing configured to be worn by the user.

In one embodiment housing includes: a first coil system housing containing the coil system; and, a second processing component housing containing signal processing components.

In one embodiment the apparatus includes a signal processor that at least partially processes the input sensor signals.

In one embodiment the signal generator includes: a driver circuit that generates controlled drive signals in accordance with signals from the controller; and, a trigger circuit for each coil that uses the drive signals to generate the stimulation signals.

In one embodiment the signal generator includes a power supply including a high voltage capacitive store that stores electrical charge for use by the trigger circuits.

In one embodiment the signal generator includes an energy recovery circuitry.

In one embodiment the apparatus includes a cooling system to cool the coils.

In one embodiment the apparatus includes a response sensor that measures a response in the subject, and wherein the controller uses response signals from the response sensor to at least one of: generates the at least one stimulation signal; and, controls a position of coils in the coil array.

In one embodiment the response sensor includes an electrical impedance tomography sensor.

In one embodiment the electrical impedance tomography sensor includes: a plurality of electrodes in contact with a tissue of the subject proximate the target region; a signal generator that applies an alternating signals to a number of the plurality of electrodes; a signal sensor that measures electrical signals on other ones of the plurality of electrodes; and, one or more impedance processing devices configured to generate a map of the target region in accordance with the measured signals.

In one embodiment the map is used to at least one of: position the at least one coil; and, control stimulation signals applied to the at least one coil.

In one embodiment the system includes: a receiving coil configured to receive stray fields generated by the coil array; and, a charging system used to charge a battery using current generated by the receiving coil.

In one embodiment the system includes a tuning circuit that tunes the receiving coil.

In one embodiment the system includes a tuning circuit controller in communication with the electronic controller that controls the tuning circuit in accordance with the at least one stimulation signal.

In one embodiment the controller generates a respective stimulation signal for each of a plurality of coils in the coil system.

In one embodiment the apparatus includes an output for providing sensory stimulation to the subject.

In one embodiment the stimulatory input is audible, and the output includes a speaker for providing auditory stimulation to the subject.

In one embodiment the controller: analyses the input sensor signals to determine one or more features; and, uses the features to generate one or more stimulation signals.

In one embodiment, for an audible sensory input, the features include at least one of: features relating to a power of the acoustic signal at different frequencies; features relating to a change in power of the acoustic signal at different frequencies; features relating to a rate of change in power of the acoustic signal at different frequencies; time domain features; spectral features; cepstral features; wavelet features; Frequency coefficients; Mel Frequency Cepstral coefficients (MFCC); Gammatone Frequency Cepstral Coefficients (GFCC); GFCC delta; and, GFCC double delta.

In one embodiment the controller uses the features and at least one computational model to generate the one or more stimulation signals, the computational model embodying relationships between the features and different stimulation signals.

In one embodiment the at least one computational model is derived using at least one of: reference responses measured for reference subjects in response to reference stimulation signals generated using different features; reference responses measured for the subject in response to reference stimulation signals generated using different features; and, a model of at least the target region of the subject obtained from a 3D scan of the subject.

In one embodiment the at least one computational model is derived by applying machine learning to the reference responses and reference stimulation signals.

In one broad form an aspect of the present invention seeks to provide a method for providing sensory stimulation to a subject, the method including: using an input to acquire input signals indicative of a stimulatory input; and, using an electronic controller operating in accordance with software instructions to: receive the input signals from the input; perform analysis of the input signals; and, use results of the analysis to cause a signal generator to generate stimulation signals, the stimulation signals being applied to a coil system to thereby generate a stimulatory electromagnetic field in a target region of the subject, the stimulatory electromagnetic field being configured to selectively activate sensory neurons to thereby stimulate the subject in accordance with the stimulatory input.

In one broad form an aspect of the present invention seeks to provide an apparatus for performing neuromodulation, the apparatus including: a signal generator; a coil system including at least one axial coil; and, an electronic controller operating in accordance with software instructions that: determines neuromodulation to be performed; and, causes the signal generator to generate modulation signals, the modulation signals being applied to the coil system to thereby generate a modulation electromagnetic field in a target region of the subject, the modulation electromagnetic field being configured to perform the neuromodulation.

In one embodiment the axial coil includes a plurality of conductors extending along an axis of a coil geometry and wherein the coil geometry has a shape that is at least one of: a cone; a hemisphere; a concave hemisphere; a convex hemisphere; and, a cylinder.

In one embodiment the controller is configured to determine the neuromodulation to be performed in accordance with at least one of: input signals received via an input; and, sensor signals received from a sensor.

In one embodiment the input includes a wireless transceiver module.

In one embodiment the controller is configured to select one of a number of defined modulation sequences stored in a memory.

In one embodiment the coil system includes at least one of: at least two coils; at least three coils; at least four coils; less than ten coils; less than eight coils; and, at least one primary coil and at least one secondary coil.

In one embodiment the coil system has a coil geometry arranged to focus electromagnetic fields from each of a plurality of coils on the target region.

In one embodiment different ones of the plurality of coils are focused on different parts of the target region.

In one embodiment the coil system includes a number of coils circumferentially spaced around an axis, the axis being coincident with the target region and the coils being arranged at an angle relative to the axis, so that ends of the coils face the target region.

In one embodiment the coil system includes at least one coil coincident with the axis.

In one embodiment at least one coil is wound from a conductor at least one of: having a cross sectional area of at least one of: at least 0.001 mm²; at least 0.01 mm²; at least 0.1 mm²; at least 1 mm²; at least 5 mm²; at least 10 mm²; less than 20 mm²; and, less than 15 mm²; having a cross sectional shape of at least one of: round; and, rectangular; and, made from: a wire; a copper wire; and, a braided wire.

In one embodiment the coil is wound about a core that is at least one of: an air core; a soft magnetic composite core; an insulated magnetic core; a laminated core; a high permeability magnetic core; and, a metal core.

In one embodiment the core has at least one of: a radius of at least one of: at least 0.2 mm; at least 0.5 mm; at least 1 mm; at least 5 mm; at least 10 mm; less than 1.5 mm; less then 10 mm; less than 15 mm; and, less than 20 mm; and, a length of at least one of: at least 0.5 mm; at least 5 mm; at least 10 mm; at least 15 mm; about 20 mm-30 mm; and, less than 40 mm.

In one embodiment apparatus includes at least one shield positioned adjacent the coil system to reduce stray fields.

In one embodiment the at least one shield includes: a diamagnetic shield; a conductive shield; a shield positioned adjacent each coil; and, a shield positioned adjacent each coil, each shield including an opening having a radius of at least one of: at least 0.2 mm; at least 0.5 mm; about 1 mm; and, less than 1.5 mm.

In one embodiment the apparatus includes a housing configured to be worn by the user.

In one embodiment housing includes: a first coil system housing containing the coil system; and, a second processing component housing containing signal processing components.

In one embodiment the apparatus includes a signal processor that at least partially processes the input sensor signals.

In one embodiment the signal generator includes: a driver circuit that generates controlled drive signals in accordance with signals from the controller; and, a trigger circuit for each coil that uses the drive signals to generate the stimulation signals.

In one embodiment the signal generator includes a power supply including a high voltage capacitive store that stores electrical charge for use by the trigger circuits.

In one embodiment the signal generator includes an energy recovery circuitry.

In one embodiment the apparatus includes a cooling system to cool the coils.

In one embodiment the apparatus includes a response sensor that measures a response in the subject, and wherein the controller uses response signals from the response sensor to at least one of: generates the at least one stimulation signal; and, controls a position of coils in the coil array.

In one embodiment the response sensor includes an electrical impedance tomography sensor.

In one embodiment the electrical impedance tomography sensor includes: a plurality of electrodes in contact with a tissue of the subject proximate the target region; a signal generator that applies an alternating signals to a number of the plurality of electrodes; a signal sensor that senses signals on other ones of the plurality of electrodes; and, one or more impedance processing devices configured to generate a map of the target region in accordance with signals from the signal sensor.

In one embodiment the map is used to at least one of: position the at least one coil; and, control signals applied to the at least one coil.

In one embodiment the system includes: a receiving coil configured to receive stray fields generated by the coil array; and, a charging system used to charge a battery using current generated by the receiving coil.

In one embodiment the system includes a tuning circuit that tunes the receiving coil.

In one embodiment the system includes a tuning circuit controller in communication with the electronic controller that controls the tuning circuit in accordance with the at least one stimulation signal.

In one embodiment the controller generates a respective stimulation signal for each of a plurality of coils in the coil system.

In one embodiment the modulation electromagnetic field is configured to provide at least one of: therapeutic stimulation to the target region of the subject; and, therapeutic inhibition to the target region of the subject.

In one embodiment the neuromodulation is configured for treating Parkinson's disease and wherein the target region includes: a subthalamic nucleus of the subject; a globus pallidus internus of the subject; a ventral intermediate nucleus of the subject; and, a pedunculopontine nucleus of the subject.

In one embodiment the neuromodulation is configured to provide therapy for essential tremor and wherein the target region includes a ventral intermediate nucleus of the subject.

In one embodiment the neuromodulation is configured to provide therapy for dystonia where the target region is a globus pallidus internus of the subject.

In one embodiment the neuromodulation is configured to provide therapy for obsessive compulsive disorder and wherein the target region includes at least one of: a ventral capsule/ventral striatum of the subject; a nucleus accumbens of the subject; and a subthalamic nucleus of the subject.

In one embodiment the neuromodulation is configured to provide pain therapy and wherein the target region is a primary motor cortex of the subject.

In one embodiment the neuromodulation is configured to provide epilepsy therapy and wherein the target region includes internal capsules and regions of a thalamus of the subject.

In one embodiment the target region includes a spinal cord of the subject and wherein the neuromodulation is configured to provide therapy for at least one of: refractory chronic pain; spinal cord injuries; failed back syndrome; complex regional pain syndrome; angina pectoris; ischemic limb pain; abdominal pain; intractable pain conditions; and overactive bladder syndrome.

In one broad form an aspect of the present invention seeks to provide a method for performing neuromodulation, the method including using an electronic controller operating in accordance with software instructions to: determine neuromodulation to be performed; and, cause a signal generator to generate modulation signals, the modulation signals being applied to a coil system including at least one axial coil configured to generate a modulation electromagnetic field in a target region of the subject, the modulation electromagnetic field being configured to perform the neuromodulation.

It will be appreciated that the broad forms of the invention and their respective features can be used in conjunction and/or independently, and reference to separate broad forms is not intended to be limiting. Furthermore, it will be appreciated that features of the method can be performed using the system or apparatus and that features of the system or apparatus can be implemented using the method.

### BRIEF DESCRIPTION OF THE DRAWINGS

Various examples and embodiments of the present invention will now be described with reference to the accompanying drawings, in which:—

FIG. 1 is a schematic diagram of an example of an apparatus for providing sensory stimulation to a subject;

FIG. 2 is a flowchart of an example of a method for providing sensory stimulation to a subject;

FIG. 3 is a schematic diagram of a specific example of an apparatus for providing sensory stimulation to a subject;

FIG. 4A is a schematic diagram of an example of the physical configuration of an apparatus for providing audible sensory stimulation;

FIG. 4B is a schematic diagram of a further example of the physical configuration of an apparatus for providing audible sensory stimulation;

FIG. 5A is a schematic diagram of an example of the physical configuration of an apparatus for providing visual sensory stimulation;

FIG. 5B is a schematic diagram of a second example of a physical configuration of an apparatus for providing visual sensory stimulation to a subject;

FIG. 6 is a flowchart of a specific example of a method for providing sensory stimulation to a subject;

FIG. 7 is a flowchart of an example of a method of generating a model;

FIGS. 8A and 8B are schematic positive and negative images showing the influence of a diamagnetic shield on the field generated by a coil;

FIG. 9A is a schematic diagram of an example coil system configuration;

FIGS. 9B and 9C are schematic diagrams illustrating the electric fields generated in the subject using the coil system of FIG. 9A;

FIG. 10A is a schematic diagram of an example of an alternative coil system configuration;

FIGS. 10B and 10C are schematic positive and negative images showing the electromagnetic fields generated by the coil system of FIG. 10A;

FIGS. 11A and 11B are schematic positive and negative images of a further example of a coil system configuration and the resulting electromagnetic fields;

FIGS. 11C and 11D are schematic positive and negative images showing the current density generated in the cochlea for the coil system configuration of FIGS. 11A and 11B;

FIGS. 12A and 12B are schematic positive and negative images of a further example of a coil system configuration and the resulting electromagnetic fields;

FIGS. 13A to 13C are schematic diagrams showing examples of the electromagnetic fields generated by example conical axial coils;

FIGS. 13D and 13E are schematic diagrams showing examples of the electromagnetic fields generated by coil arrays including a number of conical axial coils;

FIG. 13F is a schematic diagram showing an example of the electromagnetic field generated by a further example of a conical axial coil;

FIGS. 14A and 14B are schematic diagrams showing examples of the electromagnetic fields generated by example concave curved axial coils;

FIG. 15 is a schematic diagram showing an example of the electromagnetic field generated by a further example of a convex curved axial coil;

FIGS. 16A and 16B are schematic diagrams showing examples of the electromagnetic fields generated by example cylindrical axial coils with a high permeability core;

FIGS. 16C and 16D are schematic diagrams showing examples of the electromagnetic fields generated by example cylindrical axial coils without a high permeability core;

FIGS. 16E and 16F are schematic diagrams showing examples of the electromagnetic fields generated by example conical axial coils without a high permeability core;

FIG. 17A is a schematic diagram showing an example of the electromagnetic fields generated in the cochlea by an example coil array including cylindrical axial coils;

FIG. 17B is a schematic diagram showing an example of the electromagnetic fields generated in the cochlea by the coil array of FIG. 17A;

FIG. 17C is a schematic side view of the coil array of FIG. 17A in use;

FIG. 17D is a schematic front view of the coil array of FIG. 17A in use;

FIG. 18A is a schematic perspective view of an example of a headset incorporating the coil array of FIG. 17A;

FIG. 18B is a schematic perspective view of an example of a controller for the headset of FIG. 18A;

FIG. 18C is a schematic side view of the headset of FIG. 18A in use;

FIG. 19A is a schematic diagram of an example of a stray field recovery system; and,

FIG. 19B is a schematic diagram of a specific example of an apparatus for providing sensory stimulation to a subject incorporating the stray field recovery system.

### DETAILED DESCRIPTION OF THE PREFERRED EMBODIMENTS

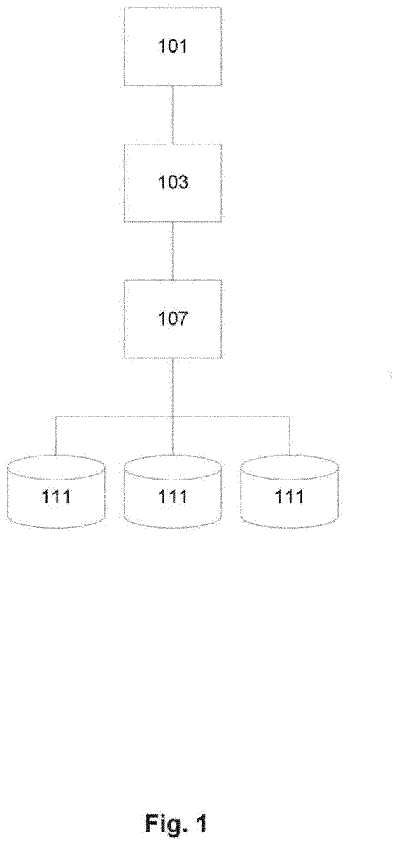

An example of an apparatus for providing sensory stimulation to a subject will now be described with reference to FIG. 1.

In this example, the apparatus includes an input 101 that acquires input signals indicative of a stimulatory input. The nature of the input will vary depending upon the preferred implementation and a wide variety of different inputs could be used. In one example, the input is in the form of a sensor that operates to sense the stimulatory input, for example using a microphone or imaging device to capture audible or visual stimulatory inputs. Alternatively, the input could be a transceiver that receives an input signal from a remote device, such as a mobile phone, or the like, as will be described in more detail below.

The apparatus includes a signal generator 107 coupled to a coil system, including one or more coils 111. The nature of the signal generator will vary depending upon the preferred implementation, but in one example the signal generator is a current source adapted to generate a varying current signal which is applied to the coils. This can include an amplifier or similar, or could include a capacitive store that can be discharged through the coils, as will be described in more detail below. The configuration of the coil system will vary depending on the sensory stimulation that is to be provided, although typically this will include a plurality of coils, such as spiral, multi-layered helical coils, or the like, optionally provided on respective cores, and further examples will be described in more detail below.

The apparatus further includes an electronic controller 103 that operates in accordance with software instructions, typically stored in a memory, or the like, and operates to receive input signals from the input 101, and control the signal generated.

The nature of the electronic controller will vary depending upon the preferred implementation but in one example the electronic controller is an integrated circuit or similar which is capable of processing input signals received from the input, analysing the input signals and controlling the signal generator. However, it will also be understood that the controller could be any electronic processing device such as a microprocessor, microchip processor, logic gate configuration, firmware optionally associated with implementing logic such as an FPGA (Field Programmable Gate Array), or any other electronic device, system or arrangement.

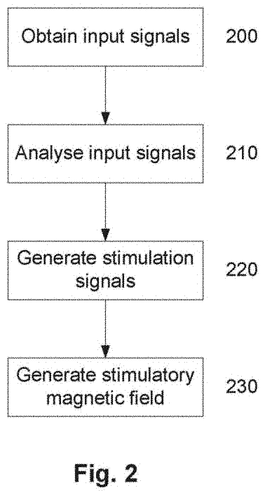

An example of operation of the apparatus of FIG. 1 will now be described with reference to FIG. 2.

In this example, at step 200 input signals are obtained by the input 101, either by sensing a sensory input, or receiving signals indicative of a sensory input from a remote device, such as a computer system, mobile phone or the like.

At step 210 the controller 103 analyses the input signals to ascertain the field(s) that needs to be generated by the coil system. The nature of the analysis that is performed will vary depending upon the preferred implementation, but typically this involves identifying particular features in the signals, such as features relating to the power of the signal at different frequencies, with these then being used either directly or indirectly to control the signal generator at step 220, thereby causing the signal to generate one or more stimulation signals.

The stimulation signals are then applied to the coil system causing the coil system to generate a stimulatory electromagnetic field at step 230. In this regard, the stimulatory electromagnetic field is generated in a target region of the subject so that it is sufficiently strong enough to activate sensory neurons in the target area, and thereby stimulate the subject in accordance with the stimulatory input.

Additionally, suitable control over the field(s) generated by the coil system, allows for localisation of the stimulatory field to target individual groups of sensory neurons, in turn allowing different responses to be obtained. For example, this can be used to stimulate different neurons in the cochlea in a manner similar to a cochlear implant, thereby allowing different audible stimulus to be reproduced.

Accordingly, the above described approach uses a electromagnetic field to stimulate sensory neurons non-invasively, using a stimulatory field of the required strength in a target area of the subject.

In this regard, a magnetic field generated by a coil is given by the Biot-Savart Law:

$$\vec{B} = \frac{μ}{4 π} I \int \frac{d \vec{l} \times \vec{R}}{{R}^{2}}$$

Where B is the magnetic flux density generated due to a coil carrying the current I, μ is the permeability of the matter and R is the displacement vector from the wire to the point where the field is calculated.

Current flow is induced in the coil, which is placed above the tissue that is being targeted, leading to a change in magnetic field. The changing magnetic field then creates an electric field given by Faraday's Law:

$$∇ \times \vec{E} = - \frac{\partial \vec{B}}{\partial t}$$

When combined, the two equations take the form:

$$∇ \times \vec{E} = - \frac{\partial \vec{B}}{\partial t} = - \frac{\partial μ \vec{H}}{\partial t} = - \frac{μ}{4 π} \frac{\partial I}{\partial t} \int \frac{d \vec{l} \times \vec{R}}{{R}^{2}}$$

Where H is the magnetic field intensity. The magnetic field can also be calculated using the magnetic vector potential:

\vec{B}=∇×\vec{A}

Where A is the magnetic vector potential. The equations then become:

$$\vec{E} = \frac{\partial \vec{A}}{\partial t} - ∇ ϕ$$

Where φ is the electrostatic potential and satisfies the Laplace equation ∇² φ=0.

The induced electric field can be split into two parts:

$${E}_{1} = \frac{\partial \vec{A}}{\partial t}$$

and E₂=∇φ. The first one is field induced due to the coil, and the second one is the field due to accumulation of charges at the tissue interface.

A neuron typically contains a lipid bilayer membrane that contains within it embedded protein structures called ion channels. These ion channels act as gateways that connect the intracellular environment (cytoplasm) to the extracellular environment. These two environments contain many charged ionic species like Na⁺, k⁺, Ca²⁺, Cl⁻ and other organic ions, the concentrations of which vary depending on the type of the ion and its presence in the intracellular or extracellular environment. Due to these different concentrations, there is a resulting potential difference between the extracellular fluids and the cytoplasm. Typically, there is an accumulation of positive charges on the extracellular side of the lipid bilayer, and negative charges on the cytoplasm side of it. This creates a potential difference between the two sides with the cytoplasm being at around 60-70 mV lower than the extracellular fluids. These ion concentrations and the potential difference are for the case when the neuron is passive, i.e. it is in a resting state, and this potential difference is called the equilibrium potential. This equilibrium potential for an ion X can be given by the Nernst Equation.

$${E}_{X} = \frac{R T}{z F} ln \frac{{[ X ]}_{o}}{{[ X ]}_{i}}$$

Where, R is the gas constant, T is the temperature given in degrees Kelvin, z is the valence of the ion, F is Faraday Constant, and [X]ₒ and [X]ᵢ are the concentrations of the ions outside and inside the cell respectively.

The expression from the Nernst Equation only provides the contribution to the membrane potential due to one type of ionic species. When combined, the potential due to a wide variety of ionic species is given by the Goldman Equation.

$${V}_{m} = \frac{RT}{F} ln \frac{{{P}_{K} [ {K}^{+} ]}_{o} + {{P}_{Na} [ {Na}^{+} ]}_{o} + {{P}_{Cl} [ {Cl}^{-} ]}_{o}}{{{P}_{K} [ {K}^{+} ]}_{i} + {{P}_{Na} [ {Na}^{+} ]}_{i} + {{P}_{Cl} [ {Cl}^{-} ]}_{i}}$$

Here, Px refers to the permeability of the membrane to that ion in units of velocity cm/s.

The ion channels can open or close due to a wide variety of triggers like voltage, mechanical force, light, and specific organic molecules, and their opening and closing comprise of the fundamental signal of neural communication called an action potential. For example, the threshold for a giant squid axon, for example, lies towards the positive side of the resting potential of −70 mV, i.e., it needs to move towards 0 mV.

Once the threshold is reached, the sodium channels opens leading to a further depolarisation of the neuron. This results in a steep rise in the membrane potential. This triggers the potassium channels to open, and since there are more potassium ions in the cytoplasm, there is an outflux of potassium ions, which polarises the cell again. There is a lag between the closing of the sodium and potassium channels, and as a result, the cell is hyperpolarised, i.e. it goes below −70 mV. It is then restored to the resting membrane potential through the ion pumps. This cascade of ion channels opening and closing results in a unique waveform of membrane potential, which is called the action potential.

In case of neural stimulation, the stimulation method aims to elicit an action potential. In case of electric and electromagnetic stimulation, the activation of a neuronal tissue is due to the interaction between the neuron and the electric field surrounding it. The electric field across a nerve fibre causes accumulation of further charges across the lipid bilayer. This leads to a potential difference, which once reaches the threshold, leads to the initiation of an action potential. For a current based stimulation, this voltage change across the membrane due to the stimulus current is given by the following equation:

$$\frac{d V}{d t} = [ - {I}_{i o n} + {I}_{s t i m u l u s} ] / {C}_{m}$$

Where V is the voltage, Iᵢₒₙ is the ion current that can be calculated from appropriate membrane models, Iₛₜᵢₘᵤₗᵤₛ is the stimulating current, and Cₘ is the membrane capacitance. In terms of the electric field, the neuronal structures can be modelled by the Hodgkin-Huxley model, and their response can be studied by the cable equation.

$$λ \frac{{\partial }^{2} V}{\partial {x}^{2}} - V - τ \frac{\partial V}{\partial t} = {λ}^{2} \frac{\partial {E}_{x}}{\partial x}$$

Where Eₓ is the axial component of the induced electric field, Vis the transmembrane voltage, λ is the space constant of the cable and τ is its time constant.

This equation however, does not take into account the ion channel dynamics. For that the equation can be altered as below.

$${λ}^{2} \frac{{\partial }^{2} V}{\partial {x}^{2}} - {g}_{L} ( {E}_{L} - V ) - {g}_{Na} ( {E}_{Na} - V ) - {g}_{K} ( {E}_{K} - V ) = {C}_{m} \frac{\partial V}{\partial t} + {λ}^{2} \frac{{\partial }^{2} A}{\partial x \partial t}$$

$$Where = \sqrt{\frac{1}{2 π {a}^{2} {r}_{i}}} ,$$

a is the axon radius, and g_{X} is the conductance of the ion channel for ion X

In the nerve equation, the value of A (vector potential) is maximised through the use of an appropriately configured coil system.

Thus, for example, for audible sensory input, inside the cochlea, the electric field is not high because the fluids of the cochlea are highly dispersive. This dispersion occurs due to their high conductivity. As a consequence, there are currents inside the cochlea that can stimulate the auditory nerve fibers in a mechanism similar to cochlear implants but the source of the currents would be changing electromagnetic fields originating outside the body instead of a current injected by an electrode placed inside the cochlea through surgery.

Accordingly, the above described apparatus operates by utilising a coil system in order to generate a stimulatory electromagnetic field in a target region of the subject. The field selectively activates sensory neurons to thereby stimulate the subject in accordance with the stimulatory input. As the coil system can be provided externally to the subject, this allows sensory stimulation of a subject without requiring an implanted device. This in turn avoids some of the disadvantages associated with implanted devices, including allowing temporary usage, avoiding damaging residual sensory perception, or the like.

The techniques can be used in a wide range of different applications, including generating visual sensory input by stimulating the retinal ganglion neurons, an optic nerve, a lateral geniculate nucleus or a visual cortex. Similarly, audible sensory input can be achieved by stimulating spiral ganglion neurons. Similar olfactory stimulation could also be generated through appropriate stimulation of the neurons in the olfactory bulb to elicit the sensation of smell in subjects with anosmia (impairment of smell), stimulation of the neurons in the taste pathways to elicit the sensation of taste, and the stimulation of vestibular neurons for people experiencing balance related ailments. The device can also be used on the somatosensory cortex to elicit the sensation of touch.

It will also be appreciated, as will be described in more detail below, that the apparatus can be adapted to provide neurostimulation more generally, and is not limited to sensory stimulation.

A number of further features will now be described.

As previously mentioned, the input can include an input sensor, such as a microphone or imaging device that senses the audible or visual stimulatory input, with other suitable sensors being used for sensing taste, smell or touch inputs.

Alternatively a wireless transceiver, such as a Wi-Fi or Bluetooth transceiver, can be provided that receives the input signal from a remote device, such as a smart phone, computer system, or the like. This can be used to avoid issues associated with attempts to detect sounds in noisy environments, bypassing the environmental noise, and allowing the user to receive sound signals directly from the remote device. This can be used in an audible context to allow a subject to engage in telephone conversations, listen to music or the like. Similar techniques could also be used for other sensory inputs, for example to allow for direct visual stimulation based on computer content, such as presentations, virtual reality feeds, or the like.

Typically the coil system is configured in order to optimise the stimulatory electromagnetic field, and preferably in order to minimise stray fields so that the magnitude of the stimulatory electromagnetic field outside the target region is minimised, whilst maximising the field strength in the target region. The manner in which this is achieved will vary depending upon the preferred implementation but this typically involves using a coil system including at least two coils, at least three coils, at least four coils, and optionally less than ten or less than eight coils, optionally including one or more primary coils and one or more secondary coils. It will be appreciated however that this is not essential and any number of coils could be used.

The use of multiple coils allows the stimulatory electromagnetic field to be generated using a superposition of a plurality of electromagnetic fields, each of which is generated using a respective coil, or a respective winding within a given coil. Using a superposition of fields is particularly advantageous, as this allows each individual field to have a lower magnitude, with a sufficiently strong stimulatory electromagnetic field only being generated in the target area where the fields overlap and constructively interfere. However, other techniques for field generation could be used, such as generating inhomogeneous electromagnetic fields and/or generating a sequence of electromagnetic fields. In this latter case, fields could be generated in a rapid sequence, so electric fields in the target region have not fully decayed before further fields are applied, with the electric fields combining to generate the required activation potential.

The coil system further has a coil geometry arranged to focus electromagnetic fields from each of the coils on the target region. This typically involves providing a number of coils circumferentially spaced around an axis that is coincident with the target region, and with the coils being arranged at an angle relative to the axis so that ends of the coils face the target region. A further central coil may also be provided coincident with the axis. This configuration maximises the strength of the stimulatory electromagnetic field in the direction and at the depth of the target region. However, it will also be appreciated that additionally and/or alternatively, different ones of the plurality of coils can be focused on different parts of the target region, allowing different sensory responses to be triggered depending on which coils are activated.

In one example, the coils may be movably mounted to a housing, to allow the coil position to be controlled dynamically, thereby ensuring the fields are focused on the target area, for example to counteract movement of a wearable device relative to the wearer. Additionally and/or alternatively, the physical configuration, including the position and orientation of the coils, would typically be determined based on a 3D scan of the subject, impedance tomography mapping, or the like, to ensure the coils generate a stimulatory electromagnetic field correctly focused on the target region of the subject.

The intensity and focus of the electromagnetic field is also dependent on the geometry of each individual coil, including the shape, number of windings, and wire diameter.

The coil typically includes a spiral, multi-layered helical coil, wound on a core, although different configurations of coils can be used depending upon the particular configuration of the coil system and the location of the target region. For example, the coil(s) could include conical tapered coils, including back-to-back tapered coils, dual lobed coils, such as figure of eight coils, butterfly coils, flat coils, spiral coils, helical coils, multi layered helical coils, or the like. The coils can be wound on a core such as an air core, a soft iron or magnetic composite core, metal core, an insulated or laminated core, a high permeability (typically over 10,000 H/m), or the like. The core can act to focus and/or strengthen the resulting electromagnetic field and typically has a radius of at least 0.2 mm, at least 0.5 mm, about 1 mm and less than 1.5 mm, and a length of at least 0.5 mm, at least 5 mm, at least 10 mm, at least 15 mm, about 20-30 mm and less than 40 mm, in order to produce a tightly focused field.

In one example, at least one winding of at least one coil has an inner radius that is at least 0.2 mm, at least 0.5 mm, at least 1 mm, at least 5 mm, at least 10 mm, and typically less than 1.5 mm, less than 10 mm, less than 15 mm and less than 20 mm. Similarly the coil typically has an outer radius of at least 5 mm, at least 8 mm, at least 10 mm, at least 20 mm, at least 300 mm, less than 50 mm and typically less than 60 mm. It is found that these dimensions are both suitable for providing the required field strength, whilst avoiding the overall coil size from being unduly large and making the resulting coil system impractical from a use perspective.

In another example, the coil system includes at least one axial coil. Axial coils typically include a plurality of conductors extending along an axis of a coil geometry, with a return path extending in a direction at least partially non-parallel to the axis, so that the coils generate an electric field in the target region. Specifically, for coils of this arrangement, the main field generated is an electric field that extends outwardly from the coil aligned with the coil axis, which can help generate a more focused and/or higher strength field in a target region. Thus, by positioning the coils with axes aligned with the target region, this can be used to create electric fields in the target region.

The coil axial coil typically includes a plurality of conductors extending along an axis of a coil geometry, with the coil geometry having a shape that is at a cone, a hemisphere, a concave hemisphere, a convex hemisphere or a cylinder, and examples will be described in more detail below.

The coil is wound from a conductor having a cross sectional area of at least one of at least 0.001 mm², at least 0.01 mm², at least 0.1 mm², at least 1 mm², at least 5 mm², at least 10 mm², about 0.05 mm², less than 20 mm² and less than 15 mm². Whilst the cross sectional area of the conductor per se has little impact on field strength, using a smaller cross sectional area conductor results in a larger number of windings, which can in turn increase the strength of the resulting electromagnetic field. This however needs to be balanced by ensuring the conductor is able to carry the required currents. Additionally, the conductor can have a round or rectangular shape, the latter of which can assist in maximising the conductor density for a given winding configuration. The conductor can be made from a wire, a copper wire, a braided wire, such as a braided Litz wire, or the like.

In a further example, the core can taper inwardly approximately an end of the core closest to the subject, which can help focus the generated electromagnetic field further, in turn helping minimise stray fields. Additional focussing can be obtained through the use of a shield formed from a diamagnetic or conductive material positioned adjacent the coil system. The shield can include a single shield but more typically includes a respective shield for each coil in the array, with the shield being positioned adjacent each coil, and including an opening having a radius of at least 0.2 mm, at least 0.5 mm, about 1 mm and less than 1.5 mm. Shielding can also be used to reduce external stray fields, which can assist in device usability.

In a further example, stray fields can be attenuated by recovering energy from the fields, which can in turn be used in order to at least partially power the apparatus, for example by charging a battery. In this example, the system further includes a receiving coil configured to receive stray fields generated by the coil array and a charging system used to charge a battery using current generated by the receiving coil.

The ability of the receiving coil to scavenge energy from the stray fields will depend on the impedance of the receiving coils and the frequency of the fields generated by the coil array. Accordingly, in one example, the system includes a tuning circuit that tunes the receiving coil, thereby optimising the recovery of energy from the stray field. In one preferred arrangement, the system includes a tuning circuit controller in communication with the electronic controller. In this example, the tuning circuit controller controls the tuning circuit in accordance with the at least one stimulation signal, so that the receiving coil and associated circuitry is optimised for the field currently being generated by the coil array.

It has been found that the above described configurations result in coils that generate a sufficiently strong and focussed electromagnetic field, but which are not unduly large from a physical perspective, allowing these to be accommodated comfortably within a housing worn by the subject.

The apparatus typically further includes a housing configured to be worn by the user. In one example, the housing is formed in two parts, including a first coil system housing containing the coil system and a second processing component housing containing signal processing components, such as the controller and a power supply, such as rechargeable or replaceable batteries.

The housing(s) is typically sealed to prevent ingress of water or other contaminants, and may be configured to conform to the subject for comfort. For example, in the case of a system for stimulatory audible responses, the housing could have a form factor similar to a pair of headphones and could include a cushion for comfort on the ear, and may include a securing means, such as a strap, headband and/or double sided adhesive tape, for firm positioning of the device on the ear.

The system can also include a digital signal processor (DSP) that at least partially processes the input signals, for example to perform filtering or the like, which can help reduce subsequent downstream processing.

The electromagnetic field induced by the coil is directly responsible for the stimulation of neurons. As the electromagnetic pulses have to pass through the skull, this creates a requirement for powerful electromagnetic fields emanating from the coil. Accordingly, in one example the signal generator includes a driver circuit that generates controlled drive signals in accordance with the signals from the controller and a trigger circuit for each coil that uses the drive signals to generate the stimulation signals. The trigger circuit can be coupled to a high voltage capacitive store that stores electrical charge so that a large current can discharged into the coils by the trigger circuit. In one example, the capacitive storage can be charged up to 2 kV and optionally have a voltage rating of up to 7.5 kV. As a result of this high voltage, the current flowing through the coil can reach up to 8000 A within several milliseconds, thereby allowing high strength electromagnetic fields to be generated, although it will be appreciated that lower currents may be used depending on the coil design.

The apparatus can include energy recovery circuitry, which allows for a quick recharge following the discharge through the coil. The powering circuits can include power electronic components like MOSFETs, Thyristors and IGBTs that act as a switch to allow for the discharge of the capacitor bank into the coil, and can transfer up to 500 J energy in less than 100 ms. The apparatus can also include circuitry that allows for the shaping of the stimulation signal pulse.

In one example, the signal generator includes a cooling system that cools the coils. The cooling system can be of any appropriate form and may include a passive cooling system, such as radiative fins that conduct heat away from the coils and/or could include a liquid based cooling system which circulates a heat transfer medium through cooling pipes provided adjacent the coils.

The apparatus can include a response sensor that measures a response in the subject, with the controller generating the stimulation signal or controlling a position of the coils, in accordance with response signals from the response sensor. Thus, the sensor can be used in order to provide feedback to help improve operation of the system, including dynamically adjusting stimulation signals, or controlling actuators to adjust a position of coils within the array. The nature of the response sensor can vary depending on the preferred implementation and could include a sensor for sensing resulting electric or electromagnetic fields, a sensor that senses a neuron response, a sensor that receives user input commands, for example to confirm a response to the stimulatory input, or the like.

In one example, the response sensor includes an electrical impedance tomography sensor, which measures tissue impedance within or surrounding the target region. In one example, the impedance tomography sensor includes a plurality of electrodes in contact with a tissue of the subject proximate the target region and a signal generator that applies an alternating signals to a number of the plurality of electrodes. A signal sensor is used to measure electrical signals on other ones of the plurality of electrodes, with one or more impedance processing devices being provided to analyse the configured to generate a map of the target region in accordance with the measured signals. The generation of such impedance maps is known in the art and will not therefore be described in any detail. In any event, once created, the map can be used to position the at least one coil and/or control stimulation signals applied to the at least one coil, thereby optimising operation of the system.

Whilst the same stimulation signal could be applied to each of the coils, more typically the controller generates a respective stimulation signal for each of the plurality of coils to thereby provide additional control over the superposed field, allowing more refined control over the activation of the sensory neurons, in turn improving the quality of the sensory stimulation. Specifically, this can include adjusting the magnitude, frequency and/or phase of the stimulatory signals applied to each of the coils, allowing parameters of the resulting stimulatory electromagnetic field to be adjusted. In particular, this can be used to adjust a different focal point for the resulting stimulatory field, which in turn allows different neurons within the cochlea to be activated, in turn allowing different audible responses to be induced. It will be appreciated that a similar approach could also be used for other stimulatory inputs.

Additionally, the apparatus can include an output for providing sensory stimulation to the subject. For example, in the case of audible stimulatory input, the output can include a speaker, allowing sounds to be played back to the user. This can be performed in order to make use of any residual sensory capability, which can in turn increase the effectiveness of the stimulation process, and the ability of the subject to accurately perceive the sensory input.

The manner in which the controller controls the signal generator will vary depending upon the preferred implementation. Typically, the controller analyses the input signals to determine one or more features with these features then being used to control the signal generator and generate the stimulation signals.

The features can be extracted using known techniques, depending on the feature selected. In one example, the features can include any one or more of features relating to a power of the acoustic signal at different frequencies, features relating to a change in power of the acoustic signal at different frequencies, features relating to a rate of change in power of the acoustic signal at different frequencies, time domain features, spectral features, cepstral features, wavelet features, Frequency coefficients, Mel Frequency Cepstral coefficients (MFCC) and Gammatone Frequency Cepstral Coefficients (GFCC), GFCC delta and GFCC double delta features. However, it will be appreciated that other features could be used, as could other techniques.

It will also be appreciated that other signal pre-processing could be performed, for pre-processing the acoustic signal by scaling, for example to emphasize or de-emphasize features, or by adding white noise, for example to adjust signal to noise characteristics.

It will also be appreciated that different features and associated processing could be used for other stimulatory inputs. For example, in the case of visual stimulation, anisotropic diffusion techniques, such as Perona-Malik diffusion could be used to reduce the image noise without affecting the critical parts of the image content. Hidden Markov models can be used to separate a multivariate signal into additive subsets, whilst image restoration using point spread function, adaptive filtering, linear filtering or the like could also be used. Additionally, neural networks, pixilation, principal component analysis, self-organising maps, and wavelets analysis could also be used.

For the process of detection of signals which are embedded in an image, pattern recognition could be performed using transform methods, feature correlation, matched filtering, or the like. In order to represent an image efficiently for human vision predictive compression, orthogonal decomposition, and Fourier representations could be used. Other encoding techniques such as edge extraction, and pattern feature codes for object recognition, could be used for spatial structures. Furthermore, when the visual systems are subjected to translations, size variations and rotation, features such as invariance coding, circular harmonic decomposition, log-polar methods, or the like, can be used.

In one example, the controller uses the features and at least one computational model to generate the one or more stimulation signals. In this instance the computational model embodies relationships between the features and the different stimulation signals and can be derived in a variety of ways. For example, the system can examine reference responses measured for one or more reference subjects, or the subject, in response to reference stimulation signals generated using different features. Additionally and/or alternatively, the system can examine a model of at least the target region of the subject obtained from a 3D scan of the subject, thereby allowing accurate targeting of the stimulatory fields within the target region of the subject.

Thus, it will be appreciated that in one example reference responses are collected from multiple subjects, and/or the current subject, in response to reference stimulation signals, with these being correlated with different features. This allows the effect of different stimulation signals to be understood, so that different stimulation signals can be applied based on different input signals, thereby allowing the required sensory response to be induced within the subject for a given stimulatory input. In one particular example, a base model can be established based on the response of a general population, with this then being customised on a per subject basis, for example taking into account results of the scan, as well as the feedback from the subject, so that the resulting fields are optimised for the particular subject.

Whilst derivation of the model could be performed manually utilising suitable statistical analysis, in practice the model is derived utilising a machine learning algorithm. In particular, this is typically performed by utilising the reference responses obtained for different stimulation signals, with this being used to train a computational model, so that the model reflects the stimulation signals that should be used to generate a desired stimulatory response. The nature of the model and the training performed can be of any appropriate form and could include any one or more of decision tree learning, random forest, logistic regression, association or learning, artificial neuron networks, deep learning, inductive logic programming, support vector machines, clustering, Bayesian networks, reinforcement learning, representation learning, similarity and metric learning, genetic algorithms, rule-based machine learning, learning classifier systems, or the like. As such schemes are known these will not be described in any further detail.

As mentioned previously, whilst the above arrangements have been described with reference to the provision of sensory stimulation to a subject, the apparatus can be used to provide neuromodulation more broadly. In this regard, the ability to provide neuromodulation is enhanced when using an axial coil, which can generate a more focused and/or higher strength field in a target region.

In this example, the apparatus can again include a signal generator and a coil system including at least one axial coil, which typically includes a plurality of conductors extending along an axis of a coil geometry, and optionally has a coil geometry in the shape of a cone, a hemisphere, a concave hemisphere, a convex hemisphere, a cylinder, or the like. An electronic controller can be provided that determines neuromodulation to be performed and then causes the signal generator to generate modulation signals, the modulation signals being applied to the coil system to thereby generate a modulation electromagnetic field in a target region of the subject, the modulation electromagnetic field being configured to perform the neuromodulation.

Thus, it will be appreciated that in this example, the apparatus does not necessarily require an input that acquires input signals indicative of a stimulatory input, but otherwise is largely similar to the apparatus described above with respect to FIG. 1, and operates in a largely similar manner, albeit without requiring input signals to be received and analysed as described above with respect to steps 200 and 210 in FIG. 2.

Instead, the neuromodulation to be performed can be determined in other manners. For example, an input such as a wireless transceiver module could be used to allow the neuromodulation to be performed to be controlled by a remote processing device, such as a computer system, smartphone, or the like. In another example, this could be performed based on sensor signals received from a sensor, or user input provided via a user interface, allowing a sensed parameter or user input to trigger the neuromodulation. In one particular example, the controller can be configured to select one of a number of defined modulation sequences stored in a memory, for example based on sensed parameters, allowing the device to be programmed with different sequences, and with an appropriate sequence being selected as needed.

The apparatus can include features similar to those previously described. For example, the apparatus can use multiple coils in the coil array, with the coil system including a coil geometry arranged to focus electromagnetic fields from each of a plurality of coils on the target region and/or different parts of the target region.

The coil system can include a number of coils circumferentially spaced around an axis, the axis being coincident with the target region and the coils being arranged at an angle relative to the axis, so that ends of the coils face the target region, optionally including one coil coincident with the axis.

The coils can be wound from a conductor having a variety of different cross sectional areas, and made from a wire, a copper wire, a braided wire, or the like. The coils can be wound about a core, such as an air core, a soft magnetic composite core, an insulated magnetic core, a laminated core, a high permeability magnetic core, a metal core, or the like.

The apparatus can include at least one shield positioned adjacent the coil system to reduce stray fields and/or could include an energy recovery system to recover energy from the stray fields.

The apparatus may include a housing configured to be worn by the user

The apparatus can include a signal generator having a driver circuit that generates controlled drive signals in accordance with signals from the controller and a trigger circuit for each coil that uses the drive signals to generate the stimulation signals. The signal generator can include a power supply including a high voltage capacitive store that stores electrical charge for use by the trigger circuits, with an energy recovery circuitry optionally being provided.

The system may include a cooling system to cool the coils.

The apparatus can also include a response sensor, such as an impedance tomography sensor, which measures a response in the subject, allowing this to be used to control stimulation signals and/or a position of coils in the coil array. It will be appreciated that this can operate in a manner similar to that described above.

Such an apparatus can be used to provide a range of different neuromodulation, including therapeutic stimulation and/or therapeutic inhibition.

For example, the apparatus can be configured for treating Parkinson's disease, in which case the target region typically includes a subthalamic nucleus of the subject, a globus pallidus internus of the subject, a ventral intermediate nucleus of the subject or a pedunculopontine nucleus of the subject.

When providing therapy for essential tremor, the target region typically includes a ventral intermediate nucleus of the subject, whereas providing therapy for dystonia can involve stimulating a globus pallidus internus of the subject.

For providing therapy for obsessive compulsive disorder, the target region typically includes a ventral capsule/ventral striatum of the subject, a nucleus accumbens of the subject or a subthalamic nucleus of the subject.

For providing pain therapy, the target region is a primary motor cortex of the subject, whereas for epilepsy therapy, the target region includes internal capsules and regions of a thalamus of the subject.

Alternatively, the target region can include a spinal cord of the subject, in which the neuromodulation is configured to provide therapy refractory chronic pain, spinal cord injuries, failed back syndrome, complex regional pain syndrome, angina pectoris, ischemic limb pain, abdominal pain, intractable pain conditions or overactive bladder syndrome.

It will also be appreciated that other therapies could additionally and/or alternatively be provided.

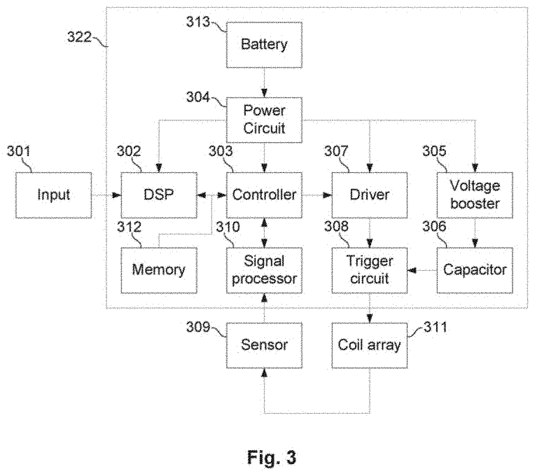

A more detailed example of a physical configuration of the apparatus will now be described with reference to FIGS. 3 to 5B, with functional components first being described with reference to FIG. 3.

In this example, the functional components typically include an input 301 such as a microphone, video camera, or the like. The input 301 is coupled to a digital signal processor 302, which is typically an integrated circuit configured to perform specific signal processing operations, such as digitising the input signal, performing frequency filtering, or the like. A processed signal is then output to controller 303, which is an electronic processing device, such as a microprocessor or similar. The controller 303 is typically coupled to a memory 312, which stores software instructions for execution by the controller 303, allowing the controller 303 to process signals and control operation of the system.

The apparatus further includes a power circuit 304 coupled to a power supply, such as a battery 313 or wireless supply, allowing power to be distributed to the digital signal processor 302, the controller 303, a driver circuit 307 and a voltage booster 305. The battery 313 could be coupled to an inductive or other charging system, allowing the battery to be charged as required. The voltage booster 305 is coupled to a capacitor 306, which operates to store charge, allowing this to be used by a trigger circuit 308 in order to generate a current that is applied to the coil system 311.

A sensor 309 is provided which measures the response signal in the subject, by measuring electrical fields within the subject's brain, with the resulting response signal undergoing processing by a signal processor 310 before being returned to the controller 303, to thereby provide feedback so that operation of the system can be adjusted to optimise performance.

The controller 303 controls the signal processors 302, 310, and sends drive control signals to the driver 307, which in turn selectively activates the trigger circuit 308 causing stimulation signals to be applied to the coils.

The apparatus can further include an amplifier and outlet, such as a speaker (not shown) in order to generate stimulation applied to the respective sensory organ, allowing any residual sense to be utilised to the extent possible. Thus, in the case of audible stimulation, an amplified version of a received audible signal can be applied to the subject's ear, so that the subject perceives an audible sensory response based on both their residual hearing and direct stimulation of their sensory neurons.

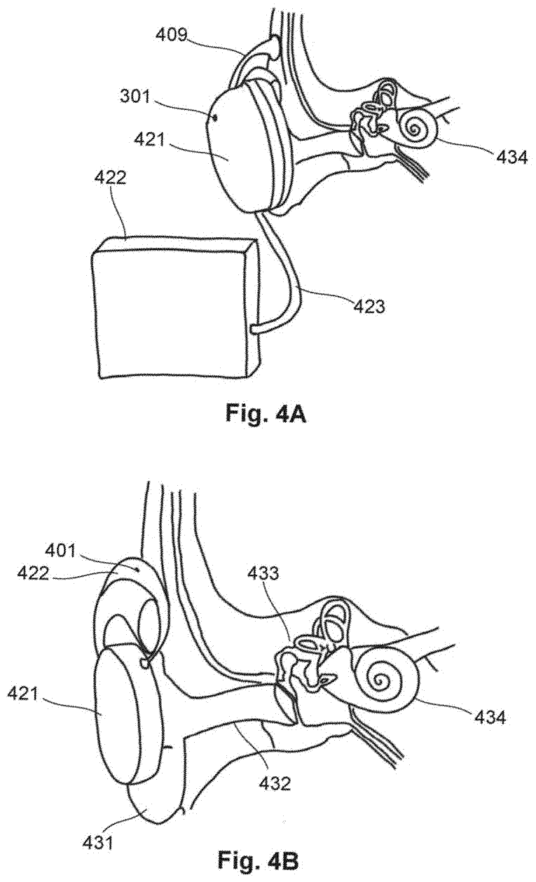

An example of physical configurations for audible sensory stimulation will now be described with reference to FIGS. 4A and 4B.

In this example, the apparatus includes a first housing 421 that accommodates the coil system 311 and the input 301. The first housing 421 is typically in the form factor of a pair of headphones or similar which can be worn by the user. A second housing 422 is provided which accommodates the electrical components, including the controller, signal generator, and the like. The components in the second housing 422 are electrically coupled to the coils 311 in the coil system via a lead 423. It will be appreciated that this configuration keeps the control electronics remote to the coils, thereby avoiding interference by the heat and electromagnetic fields generated by the coils and the processing electronics. In use the device can be positioned on the external ear 431 with the ear canal 432, middle ear 433 and cochlea 434 being provided as shown, so that the coils are able to direct the generated stimulatory field towards the spiral ganglion neurons in the cochlea.

In an alternative example, the second housing 422 is shaped to fit behind the external ear as shown in the arrangement of FIG. 4B.

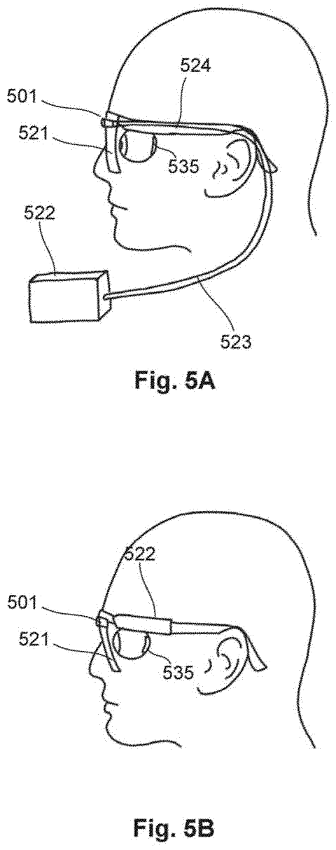

An example of a system for providing visual stimulation is shown in FIGS. 5A and 5B.

In this example, the system includes an input in the form of a camera 501 which is mounted on frames 524, which have a form factor similar to a pair of glasses. A first coil system housing 521 can project downwardly from the frame 524 in front of the eye thereby allowing the retinal ganglion neurons 535 to stimulated. Again processing electronics can be mounted in a second separate housing 522 connected via a lead 523 or could be integrated into the frame of the glasses as shown in FIG. 5B.

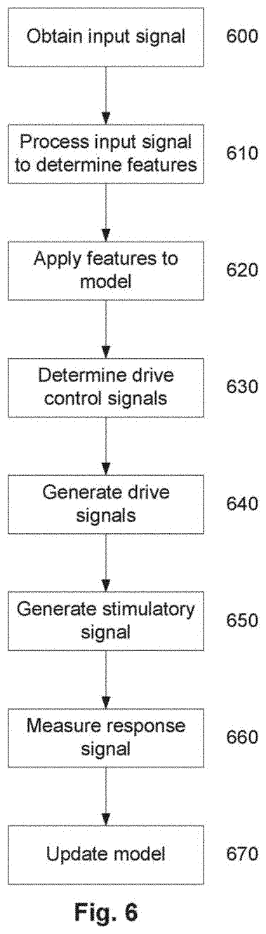

An example process for controlling the apparatus will now be described in more detail with reference to FIG. 6.

In this example, at step 600 an input signal is obtained. The input signal is obtained from the input 301, passed to the signal processor 302 and controller 303 for processing. In particular the signal processor 302 will typically perform pre-processing of the signal, for example to perform digitisation and filtering and optionally to determine spectral power features of the signal at step 610. Whilst any suitable features can be used, in one example, MFCC features are used, which are widely known in audio signal processing, especially for human speech analysis. It will be appreciated however that other signal processing techniques and features could be used for other types of stimulation, such as visual stimulation.

The process of generating the features generally involves using a filterbank to divide the frequency spectrum of the signal into multiple overlapping bands, which could be adjusted as required, and then calculating a log-energy based on a weighted sum of a Fast Fourier Transform (FFT) magnitude for each filter band. As a final step in calculating cepstral coefficients, a Discrete Cosine Transformation (DCT) is applied to the sequence of log energies, thereby yielding a number of cepstral coefficients equal to the number of filters. DCT is a standard orthogonal transformation technique that results in the most important information about the spectrum being embedded in the lower order DCT coefficients. It should be noted that the DCT coefficients capture the energy variation across the entire spectrum, for example, the first DCT coefficient is the sum of all the log-energies. It will be appreciated that other transformations, such as discrete Hartley or Hilbert Transforms, could be used, and reference to DCT is not intended to be limiting.

At step 620, the controller 303 applies the features to a computational model stored in the memory 312, with the output of the model being used to determine drive control signals at step 630, which are transferred to the driver circuit 307, which in turn generates drive signals at step 640. The drive signals are used to activate the trigger circuit 308, which in turn discharges the capacitors 306 to generate the stimulatory signals at step 650. The stimulatory signals typically have defined phases, frequencies and magnitudes, which are applied to the coils to thereby generate the necessary stimulatory electromagnetic field, in particular allowing the focal point of the field to be controlled to generate the required stimulatory response in the subject. A response signal is measured by the response sensor 309, at step 660, with this then being transferred to the controller 303 to allow the controller 303 to perform dynamic adjustment of various settings at step 670, such as to control the magnitude of signals generated in the event that insufficient stimulation is obtained, or to tune the focal point of the field, for example to accommodate changes in the physical position of the coil system housing on the subject.

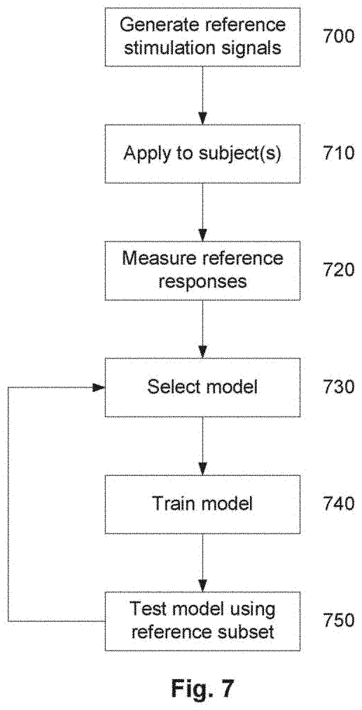

In the above described approaches, one or more computational models are used in order to determine the stimulation signals that should be generated given a respective set of input signal features. An example of a process for generating such model(s) will now be described with reference to FIG. 7.

In this example, reference stimulation signals are generated based on different features at step 700, with these being applied to reference subjects at step 710. At step 720 reference responses are determined, either measuring these using the response sensor, and/or by interrogation of the subject, to understand the sensory response they perceive.

At step 730 a model is selected, with this being trained based on the features, the applied signals and the responses at step 740. The model is used to determine a relationship between features and stimulation signals that results in a desired stimulatory response that corresponds to inputs having the respective features. This allows a stimulation signals to be generated based on one or more features derived from the input signals. The nature of the model and the training performed can be of any appropriate form and could include any one or more of decision tree learning, random forest, logistic regression, association rule learning, artificial neural networks, deep learning, inductive logic programming, support vector machines, clustering, Bayesian networks, reinforcement learning, representation learning, similarity and metric learning, genetic algorithms, rule-based machine learning, learning classifier systems, or the like. As such schemes are known, these will not be described in any further detail.

In addition to simply generating the model, the process typically includes testing the model at step 750 to assess the discriminatory performance of the trained model. Such testing is typically performed using a subset of the reference data, and in particular, different reference responses to those used to train the model, to avoid model bias. The testing is used to ensure the computational model provides sufficient accuracy.

It will be appreciated that if the model meets the accuracy requirement, it can then be used in generating the stimulation signals. Otherwise, the process returns to step 730 allowing different features and/or models to be selected, with training and testing then being repeated as required until the required discriminatory ability is obtained.

As previously mentioned, the coil system will typically also be covered with a sheet of a highly conducting metal to shield other neurons in the head from getting stimulated due to being near the wire elements of the coils in the array. The shield can be either at the edges of the whole array, or around each coil.

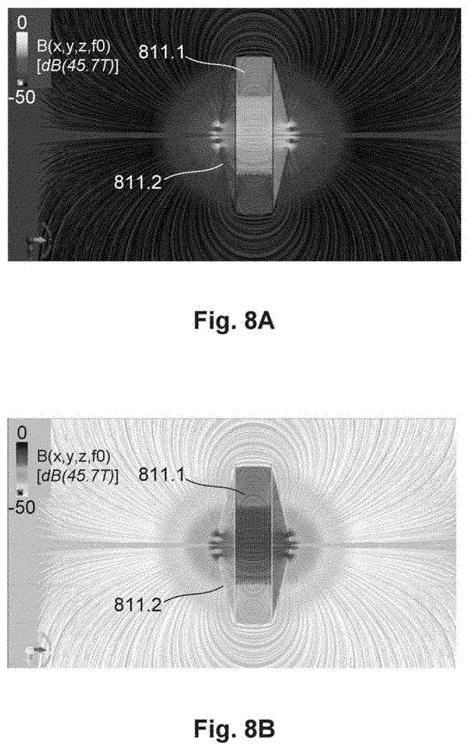

FIGS. 8A and 8B illustrate the diamagnetic effect of a shield in the form of a superconductor sheet 811.2 over a coil 811.1, which shows how the field emanating from the coil is focused at an opening of the shield 811.2. This in turn results in a more focused field within the subject, specifically allowing the fields from different coils to be highly targeted towards the relevant sensor neurons. The shield can be made of a superconductor, diamagnetic material or the like, which in one example is made of Bismuth or the like.

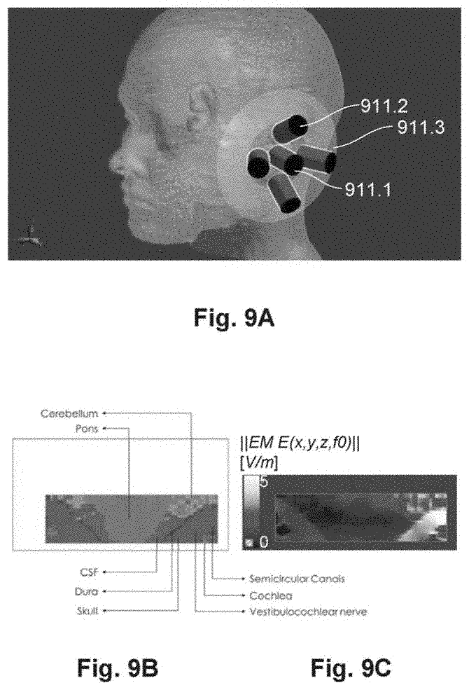

A first example coil system arrangement for generating a stimulatory electromagnetic field in the cochlea is shown in FIG. 9A.

In this example, the coil system includes five core wound coils, including a single axial coil 911.1, and four circumferentially spaced coils 911.2, each of which face the cochlea. A non-core wound surface coil 911.3 is provided against the skull around an outer circumference of the core wound coils. This design results in appropriate levels of stimulation in the cochlea nerves (About 5V/m peak electric field for stimulation), which are shown in a simulated transverse cross section of the cochlea, as shown in FIGS. 9B and 9C.

This design however, also results in a significant peripheral field in the cortex. Although the peak value of the field in the cortex is about 25V/m, which is not sufficient to initiate an action potential in cortical neurons, which typically require 100-150 V/m, reduction of the stray field is still preferred.

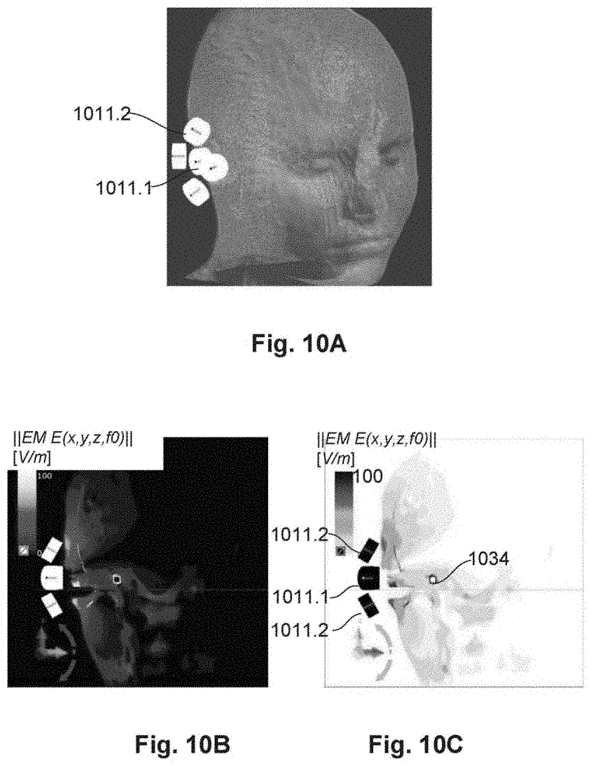

A further example is shown in FIGS. 10A to 10C.

This coil arrangement uses a five-coil system with spiral helix windings along with a ferromagnetic core and including a single axial coil 1011.1, and four circumferentially spaced coils 1011.2, each of which face the cochlea. As shown in FIGS. 10B and 10C, there are high fields in the peripheral area due to this coil geometry as well, which is not ideal.

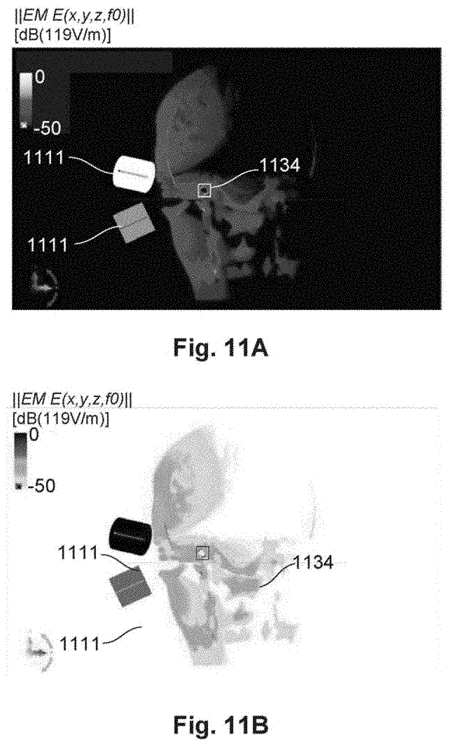

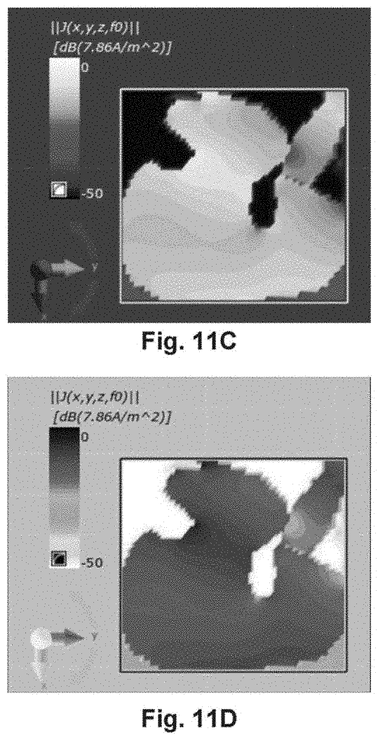

FIGS. 11A and 11B demonstrate an idealistic positioning of three coils based on finite element computational simulations.

The three coils 1111 are placed at 60 degrees from each other and the above brighter part is represented by two coils which are on the same XY plane, with the cochlea being highlighted by the white box 1134. Each coil is formed from a wire winding having a small diameter of 0.25 mm, wrapped in a spiral configuration, and extended over a helical configuration, to form a multi layered helical coil. At the center of the coil is a core with a high permeability material, which is of a diameter of 1 mm.

As it can be observed, the induced electric field is highest around the cochlea. This overcomes the disadvantage of a conventional TMS coil where the surface fields are higher than those at deeper levels inside the head. This focusing is possible due to the superposition of electromagnetic fields from each coil.

FIGS. 11C and 11D show the current levels induced in the cochlea due to the coil arrangement of FIGS. 11A and 11B. The diagram illustrates the transverse cross section of the cochlea with a top view, and shows that the current levels in the cochlea at the maxima of the J distribution are sufficient to elicit action potential in the spiral ganglion neurons and thus would lead to the sensation of hearing.

Changes in the focal point can be achieved by controlling the phase, amplitude, or frequency of the current in one or more coils in the array, thereby allowing different neurons within the cochlea to be stimulated and hence allowing different stimulatory responses to be achieved.

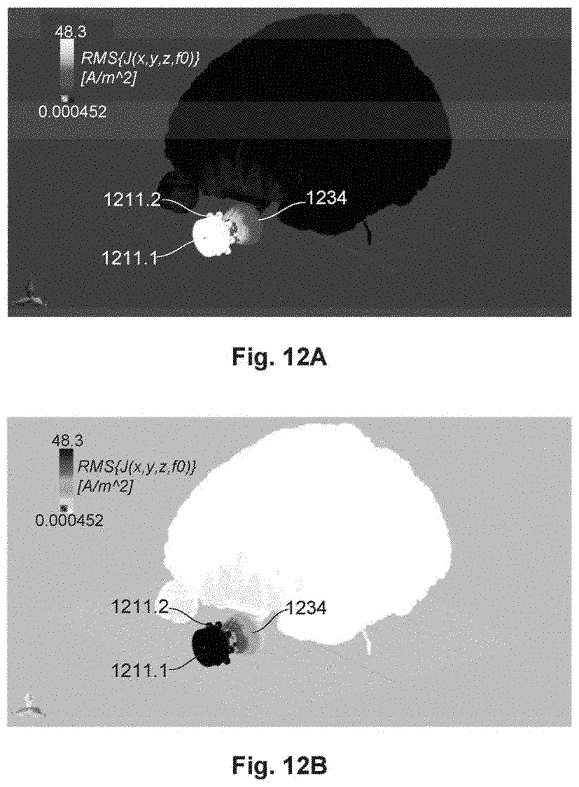

A further example arrangement for use in delivering visual stimulation is shown in FIGS. 12A and 12B.

In this example, the coil arrangement uses single central primary coil 1211.1, and a number of smaller secondary coils 1211.2 circumferentially spaced around the primary coil 1211.1 to generate fields for stimulating the retinal ganglion cells 1234.

As mentioned above, in one example, one or more axial coils can be used. In this regard, axial coils are designed to create and focus an electric field from the coil, instead of a magnetic field which is generated by the more traditional helical coil arrangements described above. As in previous design, it is the electric field that leads to the action potential, and hence direct generation of the electric field can be more effective in some circumstances.

In this regard, it is noted that for deep brain target regions the helical coils can be more effective, primarily due to the relative permeability of biological tissues being almost the same as air, and the normal component of the magnetic field on the interaction between the surfaces of two media in contact staying the same. As a result there is typically lesser attenuation of the magnetic field, which leads to a higher induced electric field at the deep brain target. In contrast, electric fields tend to attenuate faster, as the normal component of electric field changes as it goes through the boundaries of two surfaces with different dielectric properties.

It will therefore be appreciated that helical coils and axial coils might be used interchangeably for different purposes. For example, axial coils might be more effective for stimulating shallow target regions, whilst helical coils are more effective for stimulating deep target regions.

As far as the axial coil is concerned, the coil includes a plurality of conductors, formed from coil windings that pass along or adjacent to an axis of the coil geometry, with the windings extending through a periphery of the coil geometry, to maximise the current elements extending along the axis, and divert return paths away from the axis.

The fundamental equation that governs the field is the Electric field is given by:

$$\vec{{E}_{1}} = \frac{\partial \vec{A}}{\partial t}$$

When considered in conjunction with the previous analysis above, this leads to:

$$\vec{{E}_{1}} = \frac{d I}{d t} \frac{μ}{4 π} ∮ \frac{d \vec{l}}{\vert \vec{r} \vert}$$

Where d\vec{l} is the length element of the coil windings and |\vec{r}| is the distance of this element from the point of measurement of the electric field.

Thus, the axial coil allows for maximum of di elements along the axis of the coils, which produces a strong electric field in the axial direction. The part of the windings that return in order to complete the loop are placed to optimise focality and reduction of negative component of dl from them.

This coil design creates an electric field which resembles in its distribution to a magnetic field produced by a helical coil. Thus, it is an optimum design to focus a stimulating electric field. It is also possible that an embodiment of the device will utilise a combination of coils with helical winding (all the configurations mentioned in the provisional) and coils with axial windings.

Simulations have shown a focality on an area less than 1 mm². This coil design would be very effective for targets that are not too deep such as cortical regions, vagus nerve, spinal cord, retina, and perhaps the cochlea.

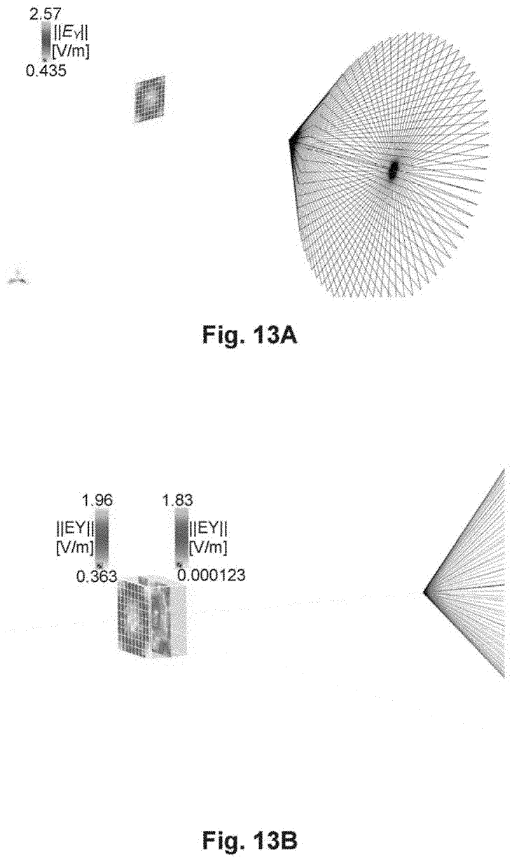

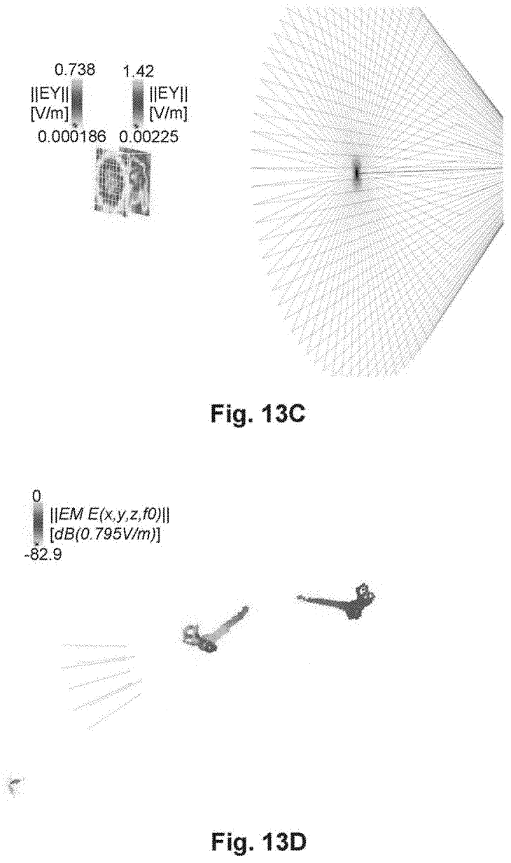

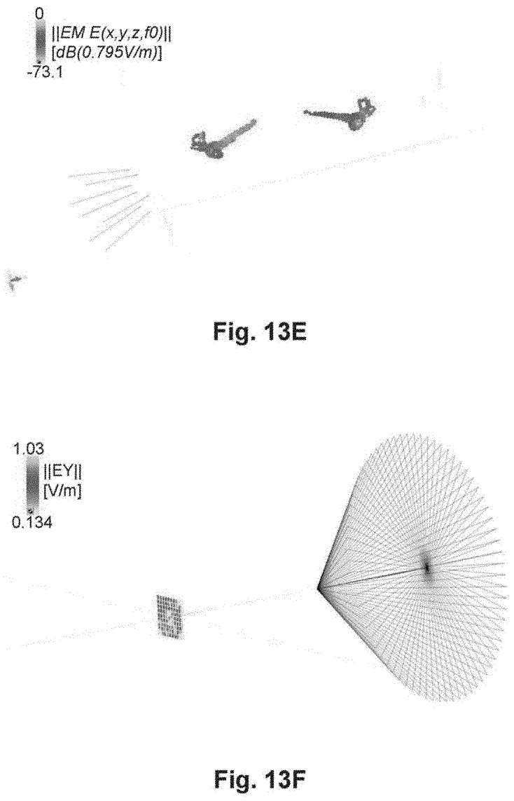

Results of simulations are shown in FIGS. 13 to 16.

For example, FIGS. 13A to 13F demonstrate that an axial coil having a conical configuration can generate a significant electrical field in a target region offset from a cone tip, whilst a field magnitude is smaller behind the coil, leading to reduced stray fields. Furthermore, coil arrays including seven conical coils can generate sufficient electric fields in the cochlea, as shown in FIGS. 13D and 13E, to allow sensory action potentials to be induced.

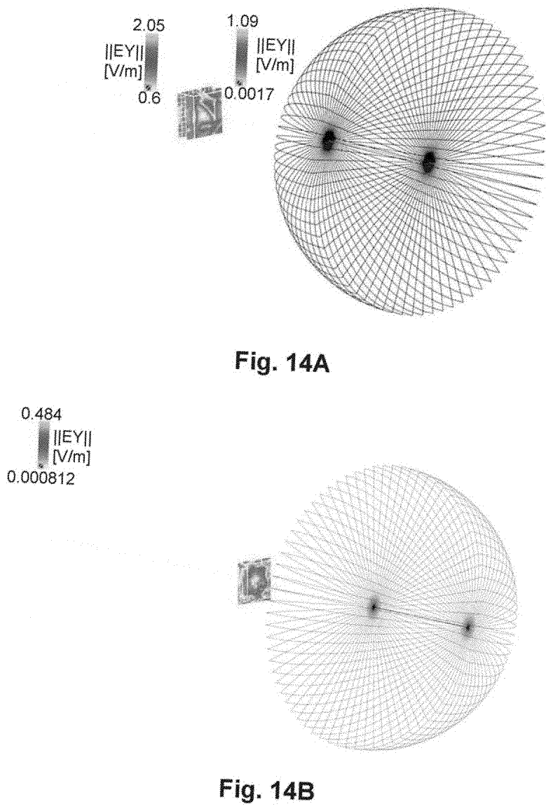

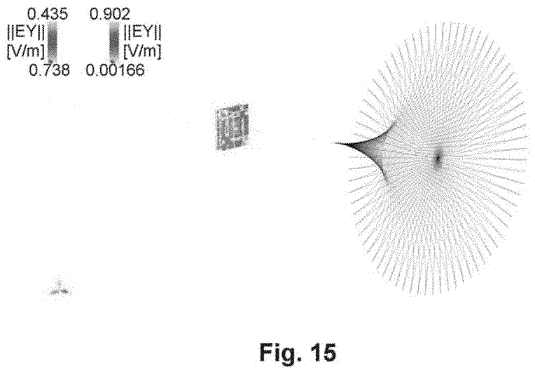

Convex and concave hemispherical coils are shown in FIGS. 14A and 14B and FIG. 15, respectively, which again generate a suitable electric field, whilst minimising stray fields.

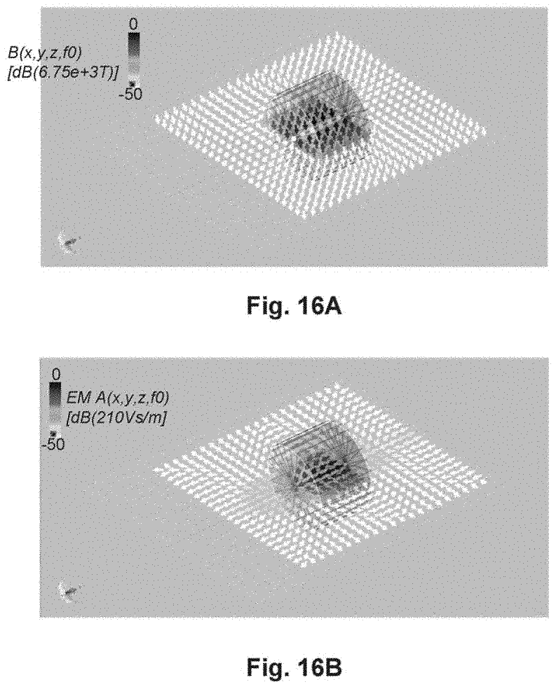

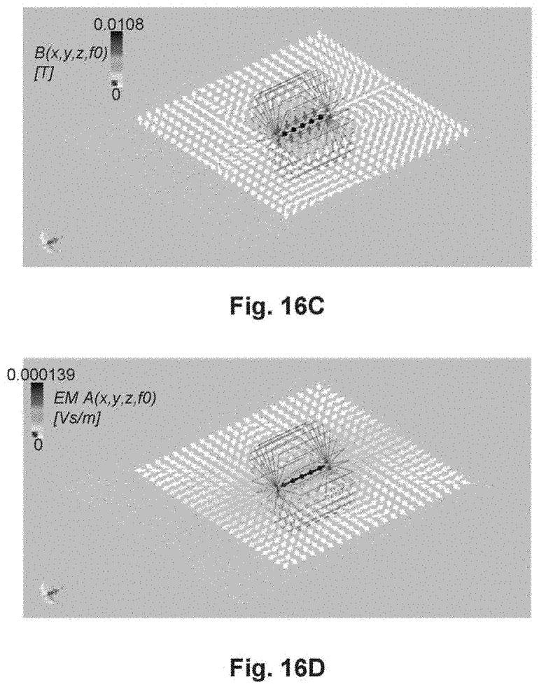

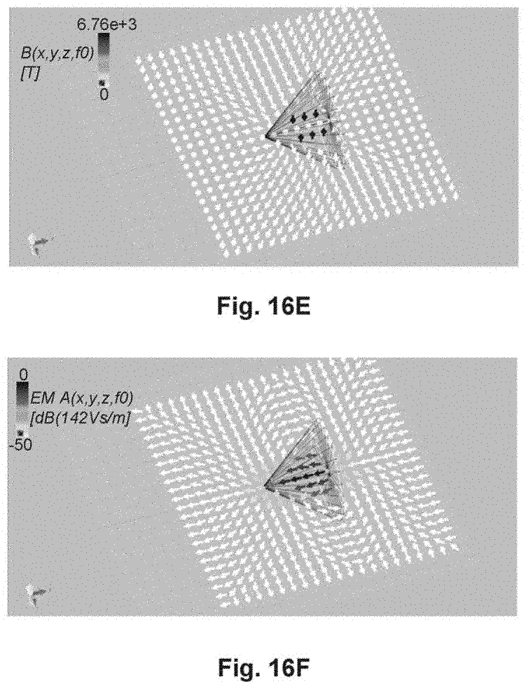

The fields generated within cylindrical axial coils with a high permeability core, cylindrical coils without a high permeability core and conical axial coils without a high permeability core are shown in FIGS. 16A and 16B, 16C and 16D, and 16E and 16F.

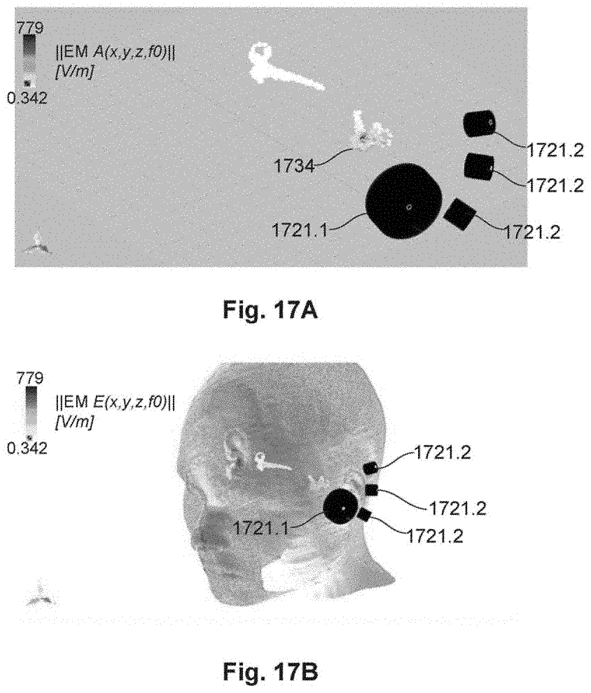

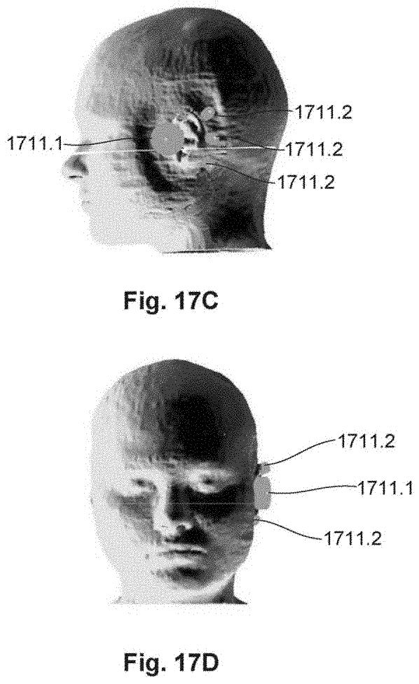

A specific example of a coil array utilising cylindrical helical coils is shown in FIGS. 17A to 17D. In this example, the coil arrangement uses single central primary cylindrical axial coil 1711.1, and a number of smaller secondary cylindrical axial coils 1711.2 circumferentially spaced around the primary coil 1711.1 to generate fields for stimulating the cochlea 1734. In this example, each coil faces the cochlea, so that the generated electromagnetic fields are focused on the cochlea, as shown by the resulting field strengths within the cochlea. It will be appreciated that similar configurations can be implemented using axial coils.

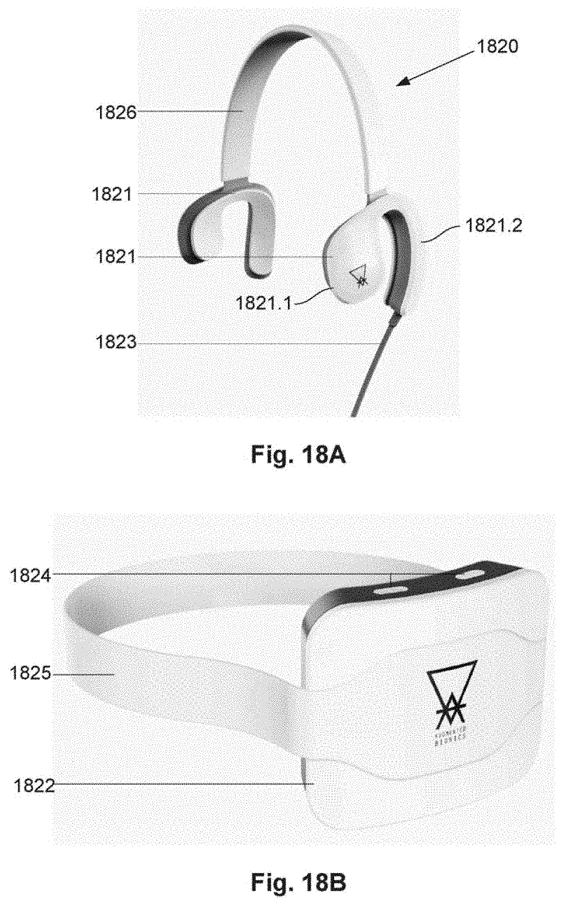

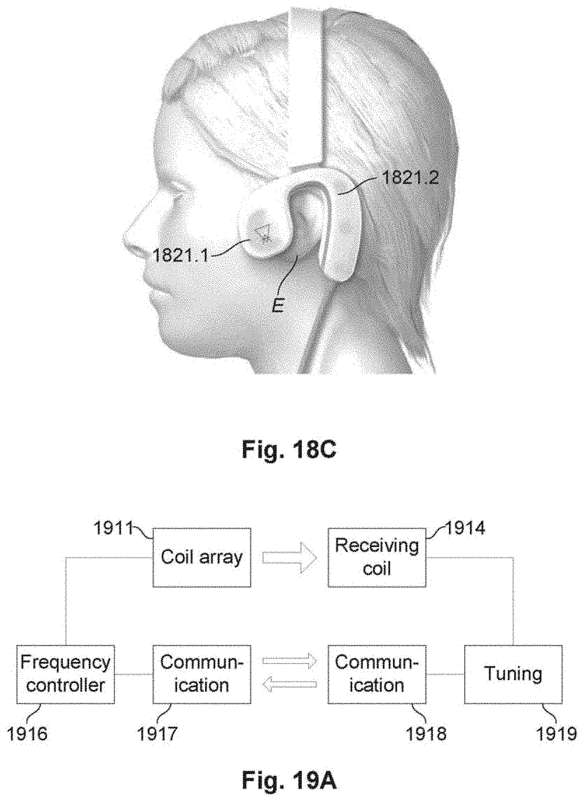

The above described coil arrangement is particularly useful, as this can be easily incorporated into housing arrangement suitable for wearing by a patient, and an example of this will now be described in more detail with reference to FIGS. 18A to 18C.

In this example, the system includes a headset 1820 including two first housings 1821, supported by a headband 1826. The first housings include front and rear lobes 1821.1, 1821.2, which sit in front and behind the subject's ear E, with the front lobe 1821.1 incorporating the primary coil 1711.1 and the rear lobe 1821.2 incorporating the secondary coils 1711.2.

A second housing 1822 is provided, which accommodates the electrical components, including the controller, signal generator, and the like. The second housing 1822 includes ports that connect to a lead 1823 extending from headset 1820 to electrically couple the coils 1711 to the control system. The second housing 1822 is coupled to a belt 1825, allowing this to be worn around the user's waist.

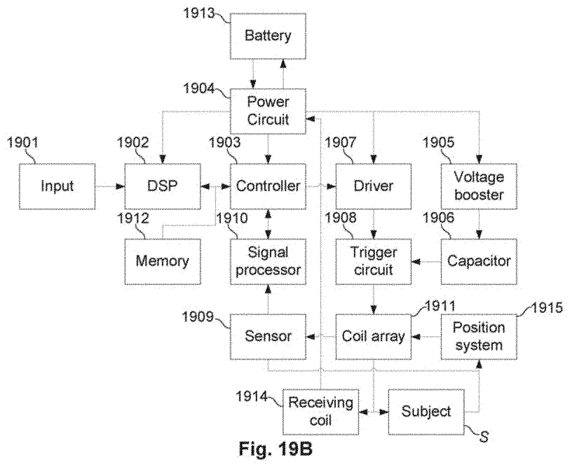

As previously mentioned, in one example the system can generate a stray field close to the stimulating coil array, which can be recovered using a wireless charging mechanism, which will now be described in more detail with reference to FIGS. 19A and 19B.

In this example, the system includes a coil array 1911 in communication with a controller 1916, which controls the frequency of stimulation signals applied via the coil array 1911. This is coupled to a first communications module 1917, which in turn communicates with a tuning system 1919 via a second communications module 1918, allowing the tuning system to be provided with information regarding the frequency of fields generated by the coil array 1911. The tuning system 1919 is coupled to a receiving coil 1914, allowing the impedance of the receiving coil 1914 to be tuned and thereby absorb as much energy as possible from the stray field.

Thus, this arrangement uses a tightly coupled magnetic wireless charging mechanism to charge the system back with the magnetic field created in the superficial region of the stimulating coil. In this scenario the transmitting coil is a high current TMS stimulating coil array 1911 and receiving coils 1914 are positioned between the primary coil and human head. Receiving coils will operate in resonant frequency of 120-140 kHz. Inductor-capacitor tank circuit is utilised to tune the frequency to increase the coupling factor. The magnetic field generated by the primary coil will be captured by the receiving coil then rectified, filtered distortions and the regulated before charging the battery.

In this example the stray electromagnetic field generated near the surface of stimulating coil array is captured by inductive charging receiving coils which is then rectified and filtered in power circuit before storing energy back in battery. The position and motion sensor such as optical position sensors or capacitive position sensors are introduced to continue monitoring the change in position of coil system and move back to the required position in the event of movement occurred.

To avoid unwanted stimulation in the event of coil displacement from its intended original position controller will temporarily shuts the coil system until position controller moves the coils back to the original position.

As mentioned above, response sensors can be provided to determine feedback about the induced action potentials. In one example, this is achieved using a impedance tomography arrangement, which generates a map showing differences in the electrical conductivity of various biological tissues. This is achieved by applying alternating currents of one or multiple frequencies through electrodes, with a number of other electrodes measuring the resulting current or voltage induced within the subject. This allows maps of equi-potentials to be used to create a 3D map of the target area. This system can enable the correct positioning of the various embodiments of the device, as changes in position of target tissues in the maps can be measured. This feedback can be either used to reposition the device before activation if the deviation is too high, or change the amount of electromagnetic fields emanating from the coils which would re-focus the fields on the target area in case of a small deviation.

An example of a control system including a charging system and response sensor system is shown in FIG. 19B.

In this example, the system includes an input 1901 such as a microphone, video camera, or the like. The input 1901 is coupled to a digital signal processor 1902, which is typically an integrated circuit configured to perform specific signal processing operations, such as digitising the input signal, performing frequency filtering, or the like. A processed signal is then output to controller 1903, which is an electronic processing device, such as a microprocessor or similar. The controller 1903 is typically coupled to a memory 1912, which stores software instructions for execution by the controller 1903, allowing the controller 1903 to process signals and control operation of the system.

The apparatus further includes a power circuit 1904 coupled to a power supply, such as a battery 1913 or wireless supply, allowing power to be distributed to the digital signal processor 1902, the controller 1903, a driver circuit 1907 and a voltage booster 1905. The battery 1913 could be coupled to an inductive or other charging system, allowing the battery to be charged as required. In additional, the power circuit 1904 is coupled to a receiving coil 1914, allowing recovered energy to be used to recharge the batter. A tuning circuit (not shown) would be provided as described in FIG. 19A, allowing the receiving coil to be tuned as described above. The voltage booster 1905 is coupled to a capacitor 1906, which operates to store charge, allowing this to be used by a trigger circuit 1908 in order to generate a current that is applied to the coil system 1911.

Impedance tomography sensor 1909 is provided which measures the response signal in the subject, by measuring electrical fields within the subject's brain, with the resulting response signal undergoing processing by a signal processor 1910 before being returned to the controller 1903, to thereby provide feedback so that operation of the system can be adjusted to optimise performance. Additionally, a position and control system 1915 can be used to adjust positioning of the coils in the coil array, and/or further refine control of the generated fields.

The controller 1903 controls the signal processors 1902, 1910, and sends drive control signals to the driver 1907, which in turn selectively activates the trigger circuit 1908 causing stimulation signals to be applied to the coils.

The apparatus can further include an amplifier and outlet, such as a speaker (not shown) in order to generate stimulation applied to the respective sensory organ, allowing any residual sense to be utilised to the extent possible. Thus, in the case of audible stimulation, an amplified version of a received audible signal can be applied to the subject's ear, so that the subject perceives an audible sensory response based on both their residual hearing and direct stimulation of their sensory neurons.

Accordingly, the above described system provides for the non-invasive stimulation of sensory neurons using electromagnetic pulses for the purpose of bypassing natural sensory mechanisms of the body in cases of sensory impairment such as loss of hearing, vision, smell, taste, touch, or balance.

A cochlear implant usually contains multiple electrodes placed along the spiral of the cochlea. Each electrode targets a specific audio frequency range. This relates to the tonotopic mapping of the cochlea where the auditory information at 20,000 Hz is encoded at the base of the spiral and that of 20 Hz is encoded at the tip of the spiral. The electrodes of the cochlear implant are positioned so that they target the frequency ranges most associated with human voice leading to a better understanding of speech for the users. Since the cochlea fluids are conductive, the electrodes are activated one at a time. The above system utilises a similar strategy, using multiple coils in the array to generate fields that target different locations in the cochlea at different times to thereby generate a similar response. The system contains a coil system placed lateral (adjacent) to the external ear. Each coil in the array is focused at the cochlea, with each coil being designed to produce the maximum amount of electromagnetic field for a given spatial constraint on the coil geometry. This allows a superposed field to be created within the cochlea, with a focal point of peak field strength being movable, to allow different neurons to be stimulated, in a manner similar to that achieved by a cochlear implant, but without requiring the presence of electrodes within the cochlea.

Thus, the above described arrangements can be used to provide a non-invasive hearing device. It will also be appreciated, however, that the techniques can be applied more broadly as an overall non-invasive sensory prosthesis. For example, the same overall device can also be used for the non-invasive stimulation of retinal neurons, i.e. the retinal ganglion cells, which would enable people and other mammals with loss of vision due to causes in the periphery of the retinal neurons to see again. Similarly, this could be used to stimulate other senses, including touch, smell and taste.

Beyond a prosthesis, the device can also be used for augmented and virtual reality experiences, for example to provide additional sensory experiences in virtual reality. Hence, the input could include a microphone, camera, Bluetooth, wi-fi, a wireless telemetry system coupled with a discriminator, or an electronic chemical sensor for smell and taste.

As also described above, as the system is capable of generating electromagnetic fields within a target region of the subject, the system can be used for neuromodulation more widely, and examples of this will now be described.

The US Food and Drug Administration (FDA) have approved neurostimulatory therapies for several disease states, which may be rehabilitated by stimulating the brain via deep brain stimulation, the motor cortex, the spinal cord and the vagus nerve.

Deep Brain Stimulation techniques have been FDA approved for treating medically refractory Parkinson's Disease, essential tremor, dystonia and obsessive-compulsive disorder (OCD). Other disease states are also under investigation, including Tourette's syndrome, treatment-resistant depression, chronic pain, alcohol and drug addiction, cluster headaches and Alzheimer's disease.

For deep brain stimulation, there are several adverse events associated with FDA approved neurostimulation therapies. These include but are not limited to worsened gait disturbances; dysarthria; dysphagia; neuropsychiatric and cognitive symptoms (near sensorimotor, associative and limbic functions; and medically refractory psychiatric and advanced neurodegenerative disorders, which often have comorbidities such as severe depression and cognitive deficits.

Parkinson's Disease (PD) is characterized as cardinal symptoms of tremor, rigidity, akinesia and bradykinesia, caused by the loss of dopamine cells in substantia nigra pars compacta. PD is thought to be of pathogenetic aetiology, specifically, due to mutations in alpha-synuclein, parkin, UCHL1, DJ1, PINK1, and LRRK2 genes. DJ1 and PINK1 express mitochondrial proteins involved in responses to oxidative stress and affect proteasomal function, and toxins related to the development of environment-induced PD also appear to affect the aforementioned mitochondrial functions, indicating a commonality in the factors contributing to the aetiopathogenesis of PD.

The treatment of PD is tailored to the individual patient. Currently, the optimal pharmacological therapy for PD is Levodopa, which is the natural precursor of dopamine and can be taken orally to pass the blood brain barrier. Levodopa therapy can lead to significant adverse effects such as the “wearing off” effect, levodopa-induced dyskinesias and other motor complications.

Other PD pharmacological therapeutics include catechol-o-methyl-transferase inhibitors, dopamine agonists and non-dopaminergic therapy and may be used concomitantly with levodopa or each other. The neurosurgical treatment, focusing on Deep Brain Stimulation (DBS), is also an optional therapy, however, is significantly more invasive due to the surgical nature of the intervention.

Indications for DBS therapy for PD include motor fluctuations, dyskinesia, medication-refractory tremor and medical intolerance. Symptoms that respond well to dopamine medications are also effective targets for DBS therapy, such as resting tremor, rigidity, upper extremity bradykinesia and the bradykinetic component of gait. Some symptoms are worsened by DBS, however, and these include freezing of gait (FOG), dysarthria and dysphagia.

Therefore, the ideal PD candidate for DBS therapy would exhibit L-dopa responsiveness assessed by the Unified Parkinson's Disease Rating Scale, but also dopamine non-responsive tremor. The neuro-stimulatory DBS targets for PD are: the subthalamic nucleus (STN) at high frequency (130 Hz), which improves all cardinal symptoms of PD, but is associated with a decline of specific cognitive functions (i.e. verbal fluency, learning and memory); the globulus pallidus internus (GPi) at high frequency (130 Hz), which has been shown to improve all cardinal symptoms of PD, without the greater cognitive decline of STN stimulation. Finally, the ventrolateral intermedius (VIM) can be stimulated using DBS to treat tremor symptoms of PD. As mentioned above, the FOG symptom is worsened by DBS therapy, however, lowering the frequency of stimulation to 60 Hz has produced fewer FOG episodes in PD patients. The pedunculopontine nucleus has also been demonstrated to be an effective alternative target for DBS, with reduced FOG episodes observed using this DBS target.

Essential Tremor is one of the most underlying causes of action tremor, which manifests phenotypically as the inability to control the movement of body parts while actively moving. The tremor caused by essential tremor usually stays mild and stable more many years, however, may slowly worsen over time. The aetiology of essential tremor is not well understood, however, it is believed to have a strong genetic component with approximately 50% of people with essential tremor having a family member who also has tremor.

Treatment of essential tremor is dependant on the severity of the tremor symptom. It may range from mild, where the patient is monitored by a doctor without treatment, to more severe forms, which require medical intervention. Additionally, stress and caffeine can worsen the effects of tremor and should be avoided.

The medical interventions usually indicated for essential tremor include β-blockers (to control blood pressure), anticonvulsant medications and, if pharmacological therapeutics are ineffective, the neurosurgical route is taken usually in the form of DBS.

Candidates for DBS therapy to treat essential tremor should be restricted to those with disabling action, postural, or rest tremors that significantly impact quality of life. The optimal nerve target for DBS intervention is the VIM, however, adverse events involving the development of dysarthria paresthesias have been observed due to the dissipation of current into the thalamic nucleus, posterior to the VIM, called ventralis caudalis (somatosensory thalamus). One embodiment of our device will target the VIM to offer therapy to patients with essential tremor. The use of rTMS to stimulate this nerve will avoid the dissipation into the ventralis caudalis encountered with tDCS.

Dystonia manifests as involuntary sustained muscle contraction and repetitive twisting movements, which over time result in abnormal posture. Dystonia is classified as focal, segmental, multifocal, generalized and hemidystonia. The aetiology of dystonia is can be classified as idiopathic (primary), hereditary (secondary) and trauma/secondary effects of diseases such as parkinsonian disorders and multiple sclerosis.

Treatment of dystonia varies by classification of dystonia, however, generalized dystonia can be treated pharmacologically using medications that target the nervous system, including dopamine and anticholinergic treatment. Dystonia is also treated surgically by selective denervation of muscles, however, this has an inconsistent benefit. Physical therapies, such as muscle strengthening and stretching as well as sensory training and limb-immobilization techniques have been trialled for limb dystonia but are currently of unproven benefit. An effective therapy for many neurological disorders is the use of botulinum toxin from C. botulinum, which inhibits the release of acetylcholine in into the neuromusclular junction. Botulinum toxin, when injected into dystonic muscles, reduces muscle spasm, without systemic side effects. Botulinum is the treatment of choice for many classifications of dystonia, including cervical dystonia, blepharospasm, spasmodic dysphonia, oromandibular dystonia, and limb dystonia, as it provides long-term benefit to 70-90% of patients.

DBS has been proposed as a novel therapy for dystonia, with the GPi being the target of choice. The effectiveness of GPi neurostimulation for the treatment of dystopia has been evaluated. Despite the optic tract positioned just ventral to the GPi which can lead to visual defects if the electrode is not inserted at the right depth, the FDA approved GPi-DBS for the treatment of dystopia in 2003 via the HDE pathway.

Obsessive Compulsive Disorder (OCD) can be described as intrusive anxiety-generating thoughts (obsessions) with repetitive behaviour or rituals (compulsions) perceived by the patient as necessary to reduce anxiety. OCD appears to be of complex, multifactorial aetiology. Neuroimaging studies have demonstrated that neuropathology of the basal-ganglia thalamocortical (BGTC) pathways, more specifically, in the prefrontal and limbic BGTC pathways.

OCD is usually treated pharmacologically using serotonin reuptake inhibitors (SRIs), which has shown to be largely effective in adults and moderately effective in children. Even with SRI medication, most treatment responders experience residual symptoms and are likely to relapse. Cognitive Behavioural Therapy (CBT) is also used as a form of psychological treatment and has shown to be superior to pharmacological therapies. Interestingly, DBS has shown promise in managing the symptoms of OCD, with bilateral DBS showing the highest effectiveness.

The neuro-stimulatory DBS targets for OCD are: the ventral capsule/ventral striatum (VC/VS), which is related to mood alterations and was approved by the FDA in 2003 through the HDE pathway; the nucleus accumbens (NAc); the STN; and the inferior thalamic peduncle.

Motor Cortex Stimulation (MCS) is performed alleviate patients of pain symptoms. By stimulating the primary motor cortex using MCS, individuals were alleviated of chronic pain. Interestingly, MCS of the somatosensory cortex, which is located anterior to the primary motor cortex, was observed to increase experienced pain in individuals. MCS has demonstrated to be in effective therapy for pain relief in patients with medically intractable pain, demonstrating a significant reduction in pain for neuropathic facial pain and poststroke pain (84% of patients experienced a >40% reduction in pain symptoms.

Epilepsy is a neurologic disorder that results in regularly occurring seizures, (partial or generalized) and can manifest in various ways, ranging from a person having a blank stare for a few seconds to incapacitating convulsions and loss of consciousness. Up to 30% of patients have treatment-refractory seizures that are unresponsive to antiepileptic drugs.

For these people, only neurosurgical interventions can reduce or remove seizure activity. This surgery involves the removal of problematic brain regions and is clearly highly invasive. DBS has been proposed as an alternative therapy to neurosurgery, as it is reversible and has demonstrated to significantly reduce the frequency of seizures. The neurostimulation targets for DBS to treat epilepsy and reduce the number of seizure episodes are the internal capsule and regions of the thalamus.

In 2013, NeuroPace RNS system was approved by the FDA for the treatment of medically refractory epilepsy. This therapy reduced seizures by 37.9% compared to control initially, and 66% over 6 years. Patients also experienced an improved quality of life and cognitive functions, however, the mechanism of action has still not been elucidated by the literature. One embodiment of our device will target the internal capsule and regions of the thalamus to reduce the number of seizures in individuals with epilepsy.

Spinal Cord Stimulation (SCS) is used as an alternative therapy for refractory chronic pain and may help counter the effects of spinal cord injuries. Indications for the use of SCS for spinal cord injuries are: failed back surgery syndrome; complex regional pain syndrome; angina pectoris; ischemic limb pain; abdominal pain. A literature review has concluded that SCS is a safe and effective therapy for various intractable pain conditions, with a 68% decrease in chronic pain and continuous pain relief over a 24-month period.

Spinal cord injury (SCI) is a damage to any part of the spinal cord or nerves at the end of the spinal canal. SCI often causes permanent changes in strength, sensation and other body functions below the site of injury. The severity of SCI is either classified as complete, i.e. loss of all feeling and ability to control movement below the injury, or incomplete, and can lead to paralysis which may be tetraplegia i.e. arms, hands, trunk, legs and pelvic organs are affected by the injury, or paraplegia where paralysis affects all or part of the trunk, legs, and pelvic organs.

Patients who undergo SCS therapy are able to regain volitional movement. A case study shown that combinational therapy of SCS and intense physical training was able to achieve almost 5 minutes of full weight bearing (with only balance assistance). After further training and calibration, the patient was able to regain some control of leg movement during stimulation. Another case study involved a paraplegic patient with a spinal cord injury at the sixth spinal segment. After SCS implantation and intense physical therapy, the patient was able to regain some task-specific volitional control of lower-limb movement.

One study has demonstrated that targeted SCS was successful in enabling voluntary control of walking in individuals who had sustained a spinal cord injury. Through an implanted pulse generator, selective spatiotemporal stimulation of the posterior roots of the lumbosacral spinal cord led to re-established adaptive control of paralysed muscles during overground walking. Locomotor performance improved further during rehabilitation, and after a few months, participants regained voluntary control over previously paralysed muscles without stimulation and could walk or cycle in ecological settings during spatiotemporal stimulation.

Overactive bladder syndrome manifests as the involuntary contraction of the pelvic floor muscles and relaxation of the urinary sphincter muscles, leading to involuntary urination. Treatment for OBS is usually pharmacological or surgical in nature, however, the surgical path is quite dangerous. Neurostimulation of the S3 foramen has been observed to be a suitable therapy, however, complications arising from the implant act as a deterrent. Alternative to the S3 foramen SCS implant is percutaneous tibial nerve stimulation (PTNS). PTNS utilizes the nerve root S4 and is implanted closer to the skin, at the tibial nerve slightly above the ankle. The implant acts to stimulate the spinal nerve L4 through S3). Magnetic neurostimulation is also used, but not by the PTNS route. The described patent will utilize magnetic stimulation via the PTNS site (pw 200 us @20 Hz for 30 mins, once a week).

Vagus Nerve Stimulation (VNS) can be used to treat epilepsy and therapy-resistant depression, although other indications for VNS are also under research.

For epilepsy, the device is programmed to provide regular intervals of on and off stimulation, typically 30 seconds on and 5 minutes off. This is thought to work by increasing blood flow and metabolism in regions that are involved in the onset of epileptic seizures, though the precise mechanism of action is still under debate. As of 2002, there have been approximately 16,000 VNS implants to treat epilepsy.

For therapy-resistant depression, current treatments are of neurosurgical nature and are therefore highly invasive. The minimally-invasive nature of VNS has generated a lot of attention in the medical community, although the efficacy of VNS to treat therapy resistant-depression is still under debate.

The vagus nerve has been successfully stimulated at and near the mastoid bone tip, at the tip of the mastoid bone, via the neck, between the sternomastoid muscle and trachea. The most effective stimulation was performed by SHAFIK and colleagues, which used 175 J/pulse at 40 Hz frequency (10 seconds on, 10 seconds off, for 20 mins online and 60 mins offline, 5 times per subject), and this stimulation paradigm had the longest effect.

Nearby to the vagus nerve is the phrenic nerve. Vagus nerve stimulation often co-stimulates the phrenic nerve, so correct positioning/waveform can be manipulated to minimize co-stimulation of the phrenic nerve, as described for example in JP2008/081479A (YOSHIHOTO).

There are many other indications that VNS may be efficacious as a therapy for, including post-op ileus, TNA-α dysfunction in Alzheimer's disease and any other inflammation-related disease (which can be modulated by VNS). The practicality of VNS, therefore, is not limited to disease states directly related to the vagus nerve, and can be used as a therapy for a variety of systemic conditions.

Post-operative ileus, or the inflammation of the small intestine, is extremely sensitive to surgery or other invasive therapies. It is best treated non-invasively using anti-inflammatory medications/targeting the vagus nerve with magnetic stimulation.

Alzheimer's Disease (AD) is a neurodegenerative disease, that is a common precursor to the development of Parkinson's Disease. It is characterized by the accumulation of beta-amyloid, intracellular neurofibrillary tangles, neuronal cell death and loss of synapses. The multifactorial aetiology AD suggests that a combined therapy may be the optimal path to take. A large proponent of the pathophysiological cycle of AD is the chronic inflammation which gives rise to the beta-amyloid protein and reduce tau protein clearing, resulting in cytokine secretion and further inflammation, worsening the progression of AD. This inflammation is mediated by microglial secretions of the cytokines interleukin-1 (IL-1) and tumour necrosis factor alpha (TNF-α). Vagus neurostimulation has improved cognitive effects in AD patients, while also improving their pathophysiological profile, however, the mechanism for these effects are yet to be elucidated.

Post-op cognitive decline (POGD) is thought to be caused by surgical trauma subsequent surgery-mediated inflammation and may be short-term to permanent. Although there is no known therapy to treat POGD, it is hypothesized that the vagus nerve may be a suitable anti-inflammatory target to magnetic stimulation, and non-invasive therapies would be optimal to prevent further inflammation from performing another surgery for electrical VNS.

Rheumatoid arthritis—is a disease with multiple aetiology, from genetics to trauma to various disease states. It is characterized by joint inflammation, and is usually treated by either physiotherapy and exercise or by disease-modifying antirheumatic drugs (DMARDS) used in combination with other drugs to manage underlying inflammation. DMARDS utilize the same pathway as neurostimulation, that is, TNFα-mediated inflammation by the vagus nerve. DMARDS have side-effects and, while usually mild in nature, can be quite significant in severity. Treating RA with a non-surgical route of therapy is optimal as surgery can induce further inflammation, worsening disease state.

Asthma or chronic obtrusive pulmonary disease (COPD) is an umbrella term defining chronic inflammation of the lungs and is usually caused by either autoimmune responses in the lungs or environments/disease states. Though there is no cure to COPD, there are several ways to manage the inflammatory symptoms, including corticosteroids and neurostimulation of the vagus nerve. Magnetic neurostimulation may be a suitable therapeutic alternative to traditional therapies.

The Sphincter of Oddi, which is responsible for bile secretion, can also be modulated using neurostimulation to induce bile production and secretion.

According to the World Health Organization (WHO), 1.3 billion people live with some form of vision impairment. 36 million of these people are legally blind. For blindness, a majority of cases (51%) is cataract, and other causes include glaucoma, age-related macular degeneration (AMD), diabetic retinopathy, and retinitis pigmentosa (RP). Different cell types are affected by these diseases, for instance the photoreceptors are degenerating in AMD and RP, while diabetic retinopathy and glaucoma affect the retinal ganglion cells. Depending on the site of disease there are different sites of implants that provide electrical stimulation to restore vision.

Different types of visual prosthesis include epiretinal, subretinal, suprachoroidal, optic nerve, LGN, and cortical implants.

The current epiretinal prostheses consist of three components, which include a camera to capture light images, a processor to transform images into patterns of electrical stimulation, and an electrode array that sits on the inner surface of the retina and stimulates the remaining cells in the inner retina.

Humayun et al. were the pioneers of epiretinal implants. The first epiretinal device implanted in patients was the Argus I which was developed by Second Sight Medical Products and was composed of 16 platinum electrodes. The next generation of the device Argus II had 60 electrodes, and has received the CE mark for commercialization in Europe.

Subretinal implants primarily target the inner nuclear layer of the retina. They are inserted below the retina and are therefore maintained between the choroid and the retina which increases the implant stability along with risk of retinal detachments. In 2001 the first implanted subretinal device was developed by Optobionics, Inc. The device consisted 5000 photodiodes arranged in a 2 mm diameter autonomous array which directly converted light into electrical stimulation.

Transchoroidal implants stimulate the retina from the outer part. The approach provides easier implantation with removed risk of retinal detachment or choroidal hemorrhage. An Australian initiative led by Bionic Vision Australia is developing suprachoroidal implants which evokes cortical activity by stimulating the retina from outside the sclera. The strategy has been efficient in different stimulation configurations such as monopolar and bipolar.

Optic nerve is also a potential target for electrical stimulation because it conveys information of the entire visual field in a very small area. It is more challenging however, to focus the stimulation as there are more than a million axons contained in a 2 mm diameter. Optic nerve prosthesis has been shown to elicit different phosphenes through a 4-contact cuff-electrode placed around the optic nerve that emits biphasic electrical pulses of varied amplitude, duration, frequency and number of pulses per phase.

The lateral geniculate nucleus (LGN) is also a potential site for visual prosthesis. It possesses the advantage of relatively simple cell segregation on an area that is larger than the retina, which allows for adaptation of image processing to the target area with a higher resolution. LGN stimulation in alert monkeys has shown the confirmed evocation of visual percepts and their spatial localization.

In cases of glaucoma and optic neuropathy, it is not possible to stimulate the retinal neurons as they are degenerated. In this case brain stimulation is the only available strategy for a visual prosthesis. Dobelle et al. were one of the first to provide a functional cortical prothesis. Their implant was placed on the surface of the visual cortex and had 64 electrodes through which one patient was able to reach 20/1200 visual acuity.

If the electrodes penetrate the cortex, the stimulation thresholds required are two to three time lower than that for superficial stimulation of the visual cortex. The Utah electrode array for example is a device that consists 100 electrodes at the tip of acute pillars. The first functional experiments of this instrument in non-human primates, which is typically used for neuronal recordings confirmed the perception of electrically elicited phosphenes.

Accordingly, it will be appreciated from the above, that the apparatus described can be used in providing

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers. As used herein and unless otherwise stated, the terms “approximately” and “about” mean±20%.

Persons skilled in the art will appreciate that numerous variations and modifications will become apparent. All such variations and modifications which become apparent to persons skilled in the art, should be considered to fall within the spirit and scope that the invention broadly appearing before described.

1) Apparatus for providing sensory stimulation to a subject, the apparatus including:
a) an input that acquires input signals indicative of a stimulatory input;
b) a signal generator;
c) a coil system including at least one coil; and,
d) an electronic controller operating in accordance with software instructions that:
i) receives the input signals from the input;
ii) performs analysis of the input signals; and,
iii) uses results of the analysis to cause the signal generator to generate stimulation signals, the stimulation signals being applied to the coil system to thereby generate a stimulatory electromagnetic field in a target region of the subject, the stimulatory electromagnetic field being configured to selectively activate sensory neurons to thereby stimulate the subject in accordance with the stimulatory input.

2) Apparatus according to claim 1, wherein the input includes:
a) an input sensor that senses the stimulatory input, wherein the input sensor includes at least one of a microphone and an imaging device; and,
b) a wireless transceiver that receives the input signal from a remote device.

3) (canceled)

4) Apparatus according to claim 1, wherein the stimulatory input is at least one of:
a) audible, and the sensory neurons are spiral ganglion neurons; and
b) visual, and the sensory neurons are at least one of:
i) retinal ganglion neurons;
ii) an optic nerve;
iii) a lateral geniculate nucleus; and
iv) a visual cortex.

5) (canceled)

6) Apparatus according to claim 1, wherein the stimulatory electromagnetic field at least one of:
a) is generated to minimise a magnitude of the stimulatory electromagnetic field outside the target region;
b) includes at least one of:
i) a superposition of a plurality of electromagnetic fields;
ii) at least one inhomogeneous electromagnetic field; and,
iii) a sequence of electromagnetic fields.

7) (canceled)

8) Apparatus according to claim 1, wherein the coil system includes at least one of:
a) at least two coils;
b) at least three coils;
c) at least four coils;
d) less than ten coils;
e) less than eight coils;
f) at least one primary coil and at least one secondary coil;
g) a coil geometry arranged to focus electromagnetic fields from each of a plurality of coils on the target region;
h) a coil geometry arranged to focus electromagnetic fields from each of a plurality of coils on the target region and wherein different ones of the plurality of coils are focused on different parts of the target region;
i) a number of coils circumferentially spaced around an axis, the axis being coincident with the target region and the coils being arranged at an angle relative to the axis, so that ends of the coils face the target region; and,
j) at least one coil that is at least one of:
i) a conical tapered coil;
ii) a dual lobe coil;
iii a butterfly coil;
iv a flat coil;
v) a spiral coil;
vi) a helical coil;
vii) a multi layered helical coil; and,
viii) wound on a core.

9) (canceled)

10) (canceled)

11) (canceled)

12) (canceled)

13) (canceled)

14) Apparatus according to claim 8, wherein at least one winding of at least one coil has at least one of:
a) an inner radius of at least one of:
i) at least 0.2 mm;
ii) at least 0.5 mm;
iii at least 1 mm;
iv) at least 5 mm;
v) at least 10 mm;
vi) less than 1.5 mm;
vii) less then 10 mm;
viii) less than 15 mm; and,
ix) less than 20 mm; and,
b) an outer radius of at least one of:
i) at least 5 mm;
ii) at least 8 mm;
iii at least 10 mm;
iv) at least 20 mm;
v) at least 30 mm; and,
vi) less than 50 mm;
vii) less than 60 mm.

15) Apparatus according to claim 1, wherein the coil system includes at least one of:
a) at least one axial coil configured to generate an electric field in the target region; and
b) at least one axial coil configured to generate an electric field in the target region wherein the axial coil includes a plurality of conductors extending along an axis of a coil geometry and wherein the coil geometry has a shape that is at least one of:
i) a cone;
ii) a hemisphere;
iii a concave hemisphere;
iv) a convex hemisphere; and,
v) a cylinder.

16) (canceled)

17) Apparatus according to claim 1, wherein at least one of:
a) at least one coil is wound from a conductor at least one of:
(i) having a cross sectional area of at least one of:
(1) at least 0.001 mm²;
(2) at least 0.01 mm²;
(3) at least 0.1 mm²;
(4) at least 1 mm²;
(5) at least 5 mm²;
(6) at least 10 mm²;
(7)
(8) less than 20 mm²; and,
(9) less than 15 mm²;
ii) having a cross sectional shape of at least one of:
round; and,
(2) rectangular; and,
iii) made from:
(1) a wire;
(2) a copper wire; and,
(3) a braided wire;
b) the coil is wound about a core that is at least one of:
i) an air core;
ii) a soft magnetic composite core;
iii an insulated magnetic core;
iv) a laminated core;
v) a high permeability magnetic core;
vi) a metal core;
vii) has at least one of:
(1) a radius of at least one of:
(a) at least 0.2 mm;
(b) at least 0.5 mm;
(c) at least 1 mm;
(d) at least 5 mm;
(e) at least 10 mm;
(f) less than 1.5 mm;
(g) less then 10 mm;
(h) less than 15 mm; and,
(i) less than 20 mm; and,
(2) a length of at least one of:
(a) at least 0.5 mm;
(b) at least 5 mm;
(c) at least 10 mm;
(d) at least 15 mm;
(e) about 20 mm-30 mm; and,
(f) less than 40 mm; and,
viii) tapers inwardly proximate an end of the core closest to the subject.

18) (canceled)

19) (canceled)

20) (canceled)

21) Apparatus according to claim 1, wherein the apparatus includes at least one of:
a) at least one shield positioned adjacent the coil system to reduce stray fields; and
b) at least one shield positioned adjacent the coil system to reduce stray fields wherein the at least one shield includes:
i) a diamagnetic shield;
ii) a conductive shield;
iii) a shield positioned adjacent each coil; and,
iv) a shield positioned adjacent each coil, each shield including an opening having a radius of at least one of:
(1) at least 0.2 mm;
(2) at least 0.5 mm;
(3) about 1 mm; and,
(4) less than 1.5 mm.

22) (canceled)

23) Apparatus according to claim 1, wherein the apparatus includes at least one of:
a) a housing configured to be worn by the user;
b) a housing including:
i) a first coil system housing containing the coil system; and,
ii) a second processing component housing containing signal processing components;
c) a signal processor that at least partially processes the input sensor signals; and,
d) a cooling system to cool the coils.

24) (canceled)

25) (canceled)

26) Apparatus according to claim 1, wherein the signal generator includes:
a) a driver circuit that generates controlled drive signals in accordance with signals from the controller; and,
b) a trigger circuit for each coil that uses the drive signals to generate the stimulation signals;
c) a power supply including a high voltage capacitive store that stores electrical charge for use by the trigger circuits; and,
d) an energy recovery circuitry.

27) (canceled)

28) (canceled)

29) (canceled)

30) Apparatus according to claim 1, wherein the apparatus includes a response sensor that measures a response in the subject, and wherein the controller uses response signals from the response sensor to at least one of:
a) generates the at least one stimulation signal; and,
b) controls a position of coils in the coil array.

31) Apparatus according to claim 30, wherein the response sensor includes an electrical impedance tomography sensor including:
a) a plurality of electrodes in contact with a tissue of the subject proximate the target region;
b) a signal generator that applies an alternating signals to a number of the plurality of electrodes;
c) a signal sensor that measures electrical signals on other ones of the plurality of electrodes; and,
d) one or more impedance processing devices configured to generate a map of the target region in accordance with the measured signals, wherein the map is used to at least one of:
i) position the at least one coil; and,
ii) control stimulation signals applied to the at least one coil.

32) (canceled)

33) (canceled)

34) Apparatus according to claim 1, wherein the system includes:
a) a receiving coil configured to receive stray fields generated by the coil array; and,
b) a charging system used to charge a battery using current generated by the receiving coil.

35) Apparatus according to claim 34, wherein the system includes:
a) a tuning circuit that tunes the receiving coil; and
b) a tuning circuit controller in communication with the electronic controller that controls the tuning circuit in accordance with the at least one stimulation signal.

36) (canceled)

37) Apparatus according to claim 1, wherein the controller at least one of:
a) generates a respective stimulation signal for each of a plurality of coils in the coil system;
b) analyses the input sensor signals to determine one or more features and uses the features to generate one or more stimulation signals; and,
c) uses the features and at least one computational model to generate the one or more stimulation signals, the computational model embodying relationships between the features and different stimulation signals the at least one computational model being derived using at least one of:
i) reference responses measured for reference subjects in response to reference stimulation signals generated using different features;
ii) reference responses measured for the subject in response to reference stimulation signals generated using different features; and,
iii) a model of at least the target region of the subject obtained from a 3D scan of the subject and,
iv) by applying machine learning to the reference responses and reference stimulation signals.

38) Apparatus according to claim 1, wherein the apparatus at least one of:
a) includes an output for providing sensory stimulation to the subject; and,
b) includes a speaker for providing auditory stimulation to the subject.

39) (canceled)

40) (canceled)

41) A system according to claim 37, wherein, for an audible sensory input, the features include at least one of:
a) features relating to a power of the acoustic signal at different frequencies;
b) features relating to a change in power of the acoustic signal at different frequencies;
c) features relating to a rate of change in power of the acoustic signal at different frequencies;
d) time domain features;
e) spectral features;
f) cepstral features;
g) wavelet features;
h) Frequency coefficients;
i) Mel Frequency Cepstral coefficients (MFCC);
f) Gammatone Frequency Cepstral Coefficients (GFCC);
k) GFCC delta; and,
l) GFCC double delta.

42) (canceled)

43) (canceled)

44) (canceled)

45) A method for providing sensory stimulation to a subject, the method including:
a) using an input to acquire input signals indicative of a stimulatory input; and,
b) using an electronic controller operating in accordance with software instructions to:
i) receive the input signals from the input;
ii) perform analysis of the input signals; and,
iii) use results of the analysis to cause a signal generator to generate stimulation signals, the stimulation signals being applied to a coil system to thereby generate a stimulatory electromagnetic field in a target region of the subject, the stimulatory electromagnetic field being configured to selectively activate sensory neurons to thereby stimulate the subject in accordance with the stimulatory input.

46) Apparatus for performing neuromodulation, the apparatus including:
a) a signal generator;
b) a coil system including at least one axial coil; and,
c) an electronic controller operating in accordance with software instructions that:
i) determines neuromodulation to be performed; and,
causes the signal generator to generate modulation signals, the modulation signals being applied to the coil system to thereby generate a modulation electromagnetic field in a target region of the subject, the modulation electromagnetic field being configured to perform the neuromodulation.

47) (canceled)

48) (canceled)

49) (canceled)

50) (canceled)

51) (canceled)

52) (canceled)

53) (canceled)

54) (canceled)

55) (canceled)

56) (canceled)

57) (canceled)

58) (canceled)

59) (canceled)

60) (canceled)

61) (canceled)

62) (canceled)

63) (canceled)

64) (canceled)

65) (canceled)

66) (canceled)

67) (canceled)

68) (canceled)

69) (canceled)

70) (canceled)

71) (canceled)

72) (canceled)

73) (canceled)

74) (canceled)

75) (canceled)

76) (canceled)

77) (canceled)

78) (canceled)

79) (canceled)

80) (canceled)

81) (canceled)

82) (canceled)

83) (canceled)

84) (canceled)
